(19) United States
(12) Patent Application Publication
(10) Pub. No.: US 2019/0265151 A1
(43) Pub. Date: Aug. 29, 2019

(54) SYSTEMS AND METHODS TO FACILITATE GENETIC RESEARCH

(71) Applicant: PACIFIC NORTHWEST DIABETES RESEARCH INSTITUTE, Seattle, WA (US)

(72) Inventors: Aimée M. Dudley, Seattle, WA (US); Gareth Cromie, Seattle, WA (US); Amy Sirr, Seattle, WA (US); Nikita Sakhanenko, Seattle, WA (US)

(73) Assignee: PACIFIC NORTHWEST DIABETES RESEARCH INSTITUTE, Seattle, WA (US)

(21) Appl. No.: 16/317,770

(22) PCT Filed: Jul. 14, 2017
(86) PCT No.: PCT/US17/42265

§ 371 Date: Jan. 14, 2019

Related U.S. Application Data
(60) Provisional application No. 62/362,708, filed on Jul. 15, 2016.

(51) Int. Cl. G01N 15/14; C12Q 1/6869; G01N 1/30

(52) U.S. Cl. CPC G01N 15/1434; G01N 2001/302; G01N 1/30; C12Q 1/6869

(57) ABSTRACT

Systems and methods that facilitate genetic research are described. The systems and methods can utilize (1) fluorescent dyes to sort tetrads from vegetative cells, dyads and dead cells; (2) natural genetic sequences to capture tetrad relationships of recombinant progeny; and (3) markers in parental organisms to identify genetic recombination events in genomic regions of interest.

### CROSS-REFERENCE TO RELATED APPLICATION

This application claims priority to U.S. Provisional Patent Application No. 62/362,708 filed on Jul. 15, 2016, which is incorporated herein by reference in its entirety as if fully set forth herein.

### FIELD OF THE DISCLOSURE

The current disclosure provides systems and methods that facilitate genetic research. The systems and methods can utilize (1) fluorescent dyes to sort tetrads from vegetative cells, dyads and dead cells; (2) patterns of natural genetic sequences to capture tetrad relationships of recombinant progeny; and/or (3) markers in parental organisms to identify genetic recombination events in offspring in genomic regions of interest.

### BACKGROUND OF THE DISCLOSURE

Meiotic mapping, a linkage-based method for analyzing the recombinant progeny of a mating cross, has long been a cornerstone of genetic research. The method is possible in a wide range of eukaryotes, including genetically facile yeasts and less tractable microorganisms, such as the filamentous fungus Neurospora crassa and the unicellular green alga Chlamydomonas reinhardtii. The approach is enabled by tetrad disruption (e.g., dissection), a technique for isolating and cultivating all of the four spores (i.e., meiotic progeny) derived from an individual tetrad. However, the throughput of the process has historically been limited by the need to isolate tetrads out of a heterogeneous population of tetrads, vegetative cells, dyads and dead cells followed by manual separation or dissection of the spores contained in a tetrad. The process is time-consuming even for experienced researchers with access to specialized equipment. Beyond the need for methods to isolate and separate spores in a high throughput manner that maintains or re-creates original spore relationships, there is also a need for methods to detect individuals that harbor genetic recombination events in genomic regions of interest.

### SUMMARY OF THE DISCLOSURE

The current disclosure provides systems and methods that improve the ability to perform genetic research. Particular embodiments provide systems and methods to quickly and efficiently isolate tetrads from vegetative cells, dyads, and dead cells. These embodiments can utilize fluorescent dyes and flow cytometry. Additional embodiments provide systems and methods to retain or re-create original spore relationships during spore analysis, including high throughput spore analysis. These embodiments rely on patterns of natural genetic sequences in an organism and do not require genetic modification of the organism (e.g., use of introduced or expressed fluorescent proteins and/or DNA-based molecular bar codes). Additional embodiments provide systems and methods to detect genetic recombination events in genomic regions of interest. These embodiments utilize markers in the genomes of parental organisms. In the parental organisms, markers do not create a detectable signal or create a first or second differential signal. If the markers in parental strains come together in an offspring, the unified markers create a detectable signal and/or a third differential signal. The detectable or third differential signal can signify the occurrence of the genetic recombination event.

Each of the described embodiments can be practiced alone or in combination with other embodiments to generate various systems and methods that improve the ability to perform genetic research. Particular embodiments and combinations of embodiments improve the ability to perform genetic research without requiring genetic modification of the organism. These embodiments and combinations can be especially useful in industries such as the food and beverage industry, where genetic modification of organisms is discouraged or even prohibited.

### BRIEF DESCRIPTION OF THE FIGURES

Many of the drawings submitted herein are better understood in color, which is not available in patent application publications at the time of filing. Applicants consider the color versions of the drawings as part of the original submission and reserve the right to present color images of the drawings in later proceedings.

FIG. 1. Depiction of disclosed system and method utilizing (1) markers in parental organisms; (2) dye to stain viable tetrads; (3) flow cytometry to sort tetrads from vegetative cells, dyads and dead cells; (4) generation of colonies from individual spores; and (5) use of patterns of natural genetic sequences to capture tetrad relationships of recombinant progeny.

FIGS. 2A, 2B. Sporulation cultures containing vegetative yeast cells (A), tetrads (B), dyads (C) and dead cells (D) stained with DiBAC4(5).

FIG. 3. DiBAC4(5) stained yeast tetrads can be isolated from vegetative cells, dyads and dead cells using flow cytometry.

FIGS. 4A and 4B. Each colony is derived from the spore of a hand-dissected tetrad. The four colonies in each column are all derived from the same individual tetrad, thus four colonies growing in a column indicates a completely viable tetrad. (3A) spores stained with fluorescent dye, DiBAC4(5); and (3B) non-stained control. The comparison shows that tetrad staining with DiBAC4(5) does not decrease spore viability.

FIG. 5. Schematic comparison of prior art bar coding (left panel) versus disclosed methods to identify tetrad relationships of spores (right panel).

FIGS. 6A, 6B. (6A) Behavior of a single chromosome during meiosis. In the initial heterozygous diploid (top) there are two copies of the “A” haplotype (light gray chromatids) and two copies of the “B” haplotype (dark gray chromatids). Centromeres are shown as circles. Note that the two “A” centromeres stay together until the second meiotic division, as do the two “B” centromeres. Spores (haploid meiotic products) shown as dotted ovals. (6B) Segregation pattern shown for 3 chromosomes. For each chromosome whether the “light gray” or “dark gray” homologs segregate to the left or to the right side at the first meiotic division occurs at random, but for each chromosome the “light gray” and “dark gray” centromeres stay together until the second meiotic division. Therefore, at each centromere the two leftward spores always have the same allele and the two rightward spores have the other allele.

FIG. 7. Comparison of delta with interaction information for 3- and 4-spore cases. All measures were computed on a simulated dataset (1461 markers and 1140 spores from 285 tetrads). The top panel shows the scatter plot of interaction information scores versus delta scores computed on all possible groups of 3 spores. Each group is colored gray (▴; red in original) if all 3 spores of the group came from the same tetrad, dark gray (▪; blue in original) if only 2 spores came from the same tetrad, and light gray (●; green in original) if all spores came from different tetrads. The bottom panel shows the scatter plot of the scores computed on all possible groups of 4 spores. Note that sign reversal occurs between the 3- and 4-way scales for both interaction information and delta.

FIG. 8. Comparison of the amount of information between three spore tetrads (II3) and their component two-spore subgroups (MI) (bottom panel) and between four spore tetrads (II4) and their component three-spore subgroups (II3) (top panel) as measured by interaction information (II). All measures were computed on the same data as in FIG. 7. In the top panel, a group is colored dark gray (▪; red in original) for groups of 3 spores from the same tetrad considered along with the remaining spore from that tetrad, gray (▴; blue in original) for groups of 3 spores from the same tetrad considered along with one spore from another tetrad and light gray (●; green in original) otherwise. In the bottom panel, a group is colored dark gray for sets of 2 spores from the same tetrad considered along with another spore from that tetrad, gray for groups of 2 spores from the same tetrad considered along with one spore from another tetrad and light gray for groups of 3 unrelated spores. Note that the scores of gray and dark gray sets are plotted in their entirety, whereas to plot the light gray sets 2 million groups were randomly selected.

FIG. 9. An exemplary method of identifying tetrad relationships from spore genomes.

FIG. 10. The exemplary method of identifying tetrad relationships of FIG. 9 show in greater detail.

FIG. 11. Sub-portions of the exemplary method of FIG. 10 shown in greater detail.

FIGS. 12A-12C. Use of markers in the genome of parents to identify genetic recombination events in a genomic region of interest during reproduction. (FIG. 12A) Placement of genetic constructs encoding components of a marker pair within the genomes of parents around a genomic region of interest. (FIG. 12B) Alignment of chromosomes, and location of marker encoding sequences if no genetic recombination event occurs in the genomic region of interest. No detectable or differential signal is created. (FIG. 12C) Alignment of chromosomes, and location of marker encoding sequences if genetic recombination event occurs in the genomic region of interest. Detectable or differential signal is created in offspring with relevant recombination event.

FIG. 13. Use of fluorescent dyes to isolate unsporulated diploids from tetrads having a recombination event when the marker is a fluorescent protein (expressed in Spore 2).

FIG. 14. A dimorphic trait within a population of Saccharomyces cerevisiae natural isolates grown on CHROMagar Candida. CHROMagar Candida (http://www.chromagar.com/) is a commercial medium typically used in clinical settings to distinguish fungal pathogens through the use of a proprietary set of colorimetric indicators.

FIG. 15. Segregation pattern of the purple and white phenotype among the progeny of a yeast cross is indicative of a monogenic trait.

FIG. 16. The development of purple color on CHROMagar Candida maps to a region on chromosome II.

FIG. 17. Fine-mapping region delineated by drug markers.

FIG. 18. Fine mapping method isolates spores with an informative recombination event.

FIG. 19. Fine mapping method maps purple trait to a single gene.

FIG. 20 depicts is a high-level diagram showing components of a data-processing system that can be used with embodiments disclosed herein.

### DETAILED DISCLOSURE

Meiotic mapping, a linkage-based method for analyzing the recombinant progeny of a mating cross, has long been a cornerstone of genetic research. The method is possible in a wide range of eukaryotes, including genetically facile yeasts and less tractable microorganisms, such as the filamentous fungus Neurospora crassa and the unicellular green alga Chlamydomonas reinhardtii. The approach is enabled by tetrad disruption (e.g., dissection), a technique for isolating and cultivating all of the four spores (i.e., meiotic progeny) derived from an individual tetrad. However, the throughput of the process has historically been limited by the need to isolate tetrads out of a heterogeneous population of tetrads, vegetative cells, dyads and dead cells followed by manual separation or dissection of the spores contained in a tetrad. The process is time-consuming even for experienced researchers with access to specialized equipment. Beyond the need for methods to isolate and separate spores in a high throughput manner that maintains or re-creates original spore relationships, there is also a need for methods to detect individuals that harbor genetic recombination events in genomic regions of interest.

The current disclosure provides systems and methods that improve the ability to perform genetic research. Particular embodiments provide systems and methods to quickly and efficiently sort (e.g., enrich for or isolate) tetrads from vegetative cells, dyads and dead cells. These embodiments can utilize fluorescent dyes and flow cytometry. In particular embodiments, the systems and methods enrich for tetrads. In particular embodiments, the systems and methods isolate tetrads. Sorted tetrads can be analyzed in bulk form (i.e., without disruption of individual spores). In particular embodiments, sorted tetrads can be disrupted and residual dye remaining on spores can be further used to enrich for or isolate spores from vegetative cells and non-digested tetrads. Spores isolated in this manner can be used to generate colonies, liquid cultures, or biochemical extracts (e.g. DNA, RNA, proteins, or metabolites) from individual spores. This approach is beneficial for, for example, random spore analysis.

Additional embodiments provide systems and methods to capture the tetrad relationships of recombinant progeny, including in high throughput spore analysis following disruption of spores from tetrads. These embodiments rely on patterns of natural genetic sequences in an organism and do not require genetic modification of the organism (e.g., use of introduced or expressed fluorescent proteins and/or DNA-based molecular bar codes). Additional embodiments provide systems and methods to detect genetic recombination events in offspring in genomic regions of interest. These embodiments utilize markers in the genomes of parental organisms. In the parental organisms, markers do not create a detectable signal or create first and/or second differential signals. If the markers in parental strains come together in an offspring, the unified marker creates a signal (or lack of signal) that is distinguishable from the signal (or lack of signal) in the original (non-recombined, parental strains). The detectable or differential signal can signify the occurrence of the genetic recombination event.

Each of the described embodiments can be practiced alone or in combination with other embodiments to generate various systems and methods that improve the ability to perform genetic research. Particular embodiments and combinations of embodiments improve the ability to perform genetic research without requiring genetic modification of the organism. These embodiments and combinations can be especially useful in industries such as the food and beverage industry, where genetic modification of organisms is discouraged or even prohibited.

Part 1. Use of Fluorescent Dyes for Tetrad Sorting, Spore Sorting, and/or Lethality Screens.

One advantage of using yeast in genetics research is that all four haploid meiotic progeny (spores) are packaged into a tetrad, allowing the genetic information of individual meiotic events to be easily followed. While sister spores from individual tetrads are often dissected by hand, techniques have also been developed that allow large numbers of progeny to be rapidly analyzed (Michelmore et al., Proc Natl Acad Sci USA. 88: 9828-9832, 1991; Segre et al., PLoS Biol 4: e256, 2006; Ehrenreich et al., Nature 464: 1039-1042, 2010; Ludlow et al., 2013 Nat Methods. 10: 671-675; Sirr et al., 2015 Genetics 199: 247-262). Such approaches have limitations, however. Hand dissection is laborious, and current bulk methods require genetic modification to introduce reporter genes (often based on Green Fluorescent Protein, GFP) that allow the separation of tetrads from unsporulated vegetative cells and the products of incomplete meiotic events (dyads).

Disclosed herein are methods that allow bulk sorting (e.g., enriching for or isolating) of tetrads from vegetative cells, dyads and dead cells, but that do not require genetic modification of the cells. These methods can also be used to sort spores following removal from the tetrad environment based on residual dye remaining at or near the surface of the spore.

Enriching for means that the sorted target (tetrads or spores) occurs at a significantly higher frequency in a sample after sorting than before sorting. For example, the frequency of the sorted target in a sample can increase by at least 50%; at least 75%; at least 100% or more between pre- and post-sort. Isolating results in a pure population of a sorted target (tetrads or spores) lacking all cell types intended to be removed by the isolation.

Particular embodiments utilize sorting of tetrads based on an optical characteristic of a dye used to stain the tetrads. In particular embodiments, optical characteristics of a dye refer to absorption and/or emission of electromagnetic radiation within the ultraviolet, visible and/or infrared spectrum.

Particular embodiments label and sort tetrads utilizing fluorescent dyes and fluorescence-activated cell sorting (FACS).

Exemplary fluorescent dyes include xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′,5′ dichloro 2′,7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzamide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, Tetramethylrhodamine, Lissamine, ROX, Napthofluorescein, and the like, as well as other examples described elsewhere herein.

Particular embodiments disclosed herein utilize vital dyes as fluorescent dyes. Vital dyes are non-toxic dyes that have historically been used to differentiate live and dead cells within a population. Vital dyes stain based on a variety of cell characteristics that differ between live and dead cells, such as membrane potential, membrane permeability and enzyme activity. Examples of vital dyes include oxonol dyes. Oxonol dyes are lipophilic, anionic molecules that selectively stain dead cells due to collapse of membrane potential. Particular examples of vital dyes include Bis-(1,3-dibutylbarbituric acid) pentamethine oxonol (also known as DiBAC₄(5); Anaspec AS-84701), calcein AM, carboxyfluorescein diacetate, copper phthalocyanine tetrasulfonate [27360-85-6], DiOC (3,3′-dihexyloxacarbocyanine iodide), Evans blue [CAS 61-73-4], gadolinium texaphyrin [156436-89-4], indocyanine green monosodium salt [CAS 3599-32-4], isosulfan blue [also known as Patent blue violet, CAS 68238-36-8], methylene blue [CAS 314-13-6], Nile red, patent blue V [CAS 3536-49-0], patent blue VF [CAS 129-17-9], propodium iodide (PI), rhodamine 123, and sulfobromophthaleine [123359-42-2].

To stain and sort tetrads from vegetative cells, dyads, and dead cells without relying on genetic modification of the cells, a mixture of such cells can be obtained and suspended in a buffer, such as 1×PBS, pH 7.4 (1 mM Potassium Phosphate monobasic, 155 mM Sodium Chloride, 3 mM Sodium Phosphate dibasic); 1×TBS=Tris buffered saline (50 mM Tris-HCl, pH7.6, 150 mM NaCl); or 200 mM Na2HPO4, 100 mM Sodium Citrate, pH 6.2. Fluorescent dye can then be added to the cell culture at a temperature and concentration and for a period of time that allows tetrad straining. In particular embodiments, room temperature is used. Appropriate concentrations of fluorescent dye can range from, for example, 0.1 μg/ml-10 μg/ml. Generally, tetrads stain quickly, such that no significant minimum incubation time is required. Over time, the fluorescent dye's intensity can decrease. If sorting is not performed soon after incubation with fluorescent dye, steps can be taken to support stain visibility and viability of the cells, for instance by keeping the stained mixture of cells in the dark and/or on ice. Also, because only staining a small portion of the sporulation culture is necessary, one can go back to the original culture and prepare additional samples for analysis. That is, the ease of the staining protocol allows repeat staining if, for example, there is a need or desire to sort more tetrads at a later time/date.

In particular embodiments, FACS sorting utilizes a flow cytometry machine, wherein cells are interrogated by a laser. Cells can be separated into droplets with differential charges (e.g., either a positive or negative charge or varying degrees of a positive or negative charge), depending on the dye that is used. Droplets can then be sorted by charge presence or degree to allow for sorting and collection of populations of cells.

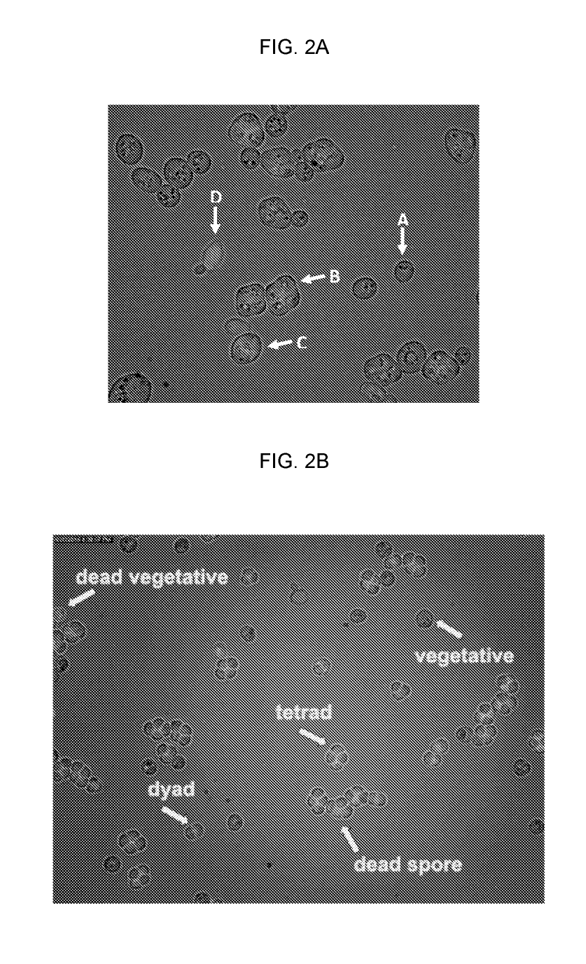

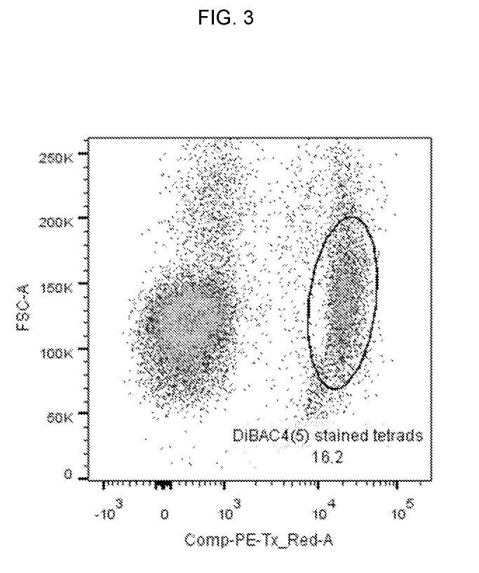

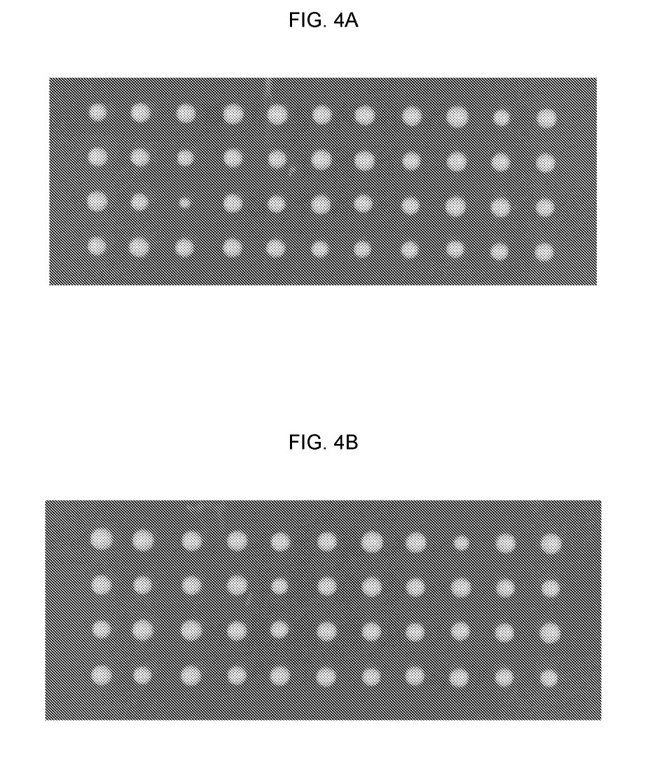

In one particular example, diploid cells were put through meiosis (sporulated) and 10⁶ cells (tetrads) were washed and resuspended in 1 ml of 1×PBS (phosphate buffered saline). DiBAC₄(5) was then added to 1 μg/ml (final concentration) and tetrads were stained at room temperature for 5 minutes prior to sorting by FACS. Red fluorescence intensity was used to sort tetrads, dyads, and dead cells away from live vegetative cells using a FACS sorter. This was accomplished using a BD FACS Aria II with 488 nm emission and 595LP 610/20 filter. FIGS. 2A, 2B show that the fluorescent dye which is substantially excluded from live cells, was retained in the region between spores of intact tetrads (the interspore region). By gating the population using forward scatter, tetrads were separated from dyads and dead cells. FIG. 3. To visualize tetrads by fluorescence microscopy (FIGS. 2A, 2B), cells were incubated for 30 minutes at 30° C. in 1 ml YPD (1% yeast extract, 2% peptone, 2% glucose) prior to staining (as described above). That staining does not affect the viability of the progeny was demonstrated by hand dissecting and growing stained (FIG. 4A) and unstained (FIG. 4B) tetrads.

FIG. 2B depicts a tetrad where one of the 4 spores is stained in a way that indicates that it is dead. Thus, particular embodiments can also be used to detect the genetic phenomenon of synthetic lethality. The ability to isolate (e.g., by FACS) a population of tetrads where one or more of the spores are dead provides a novel method for performing synthetic lethality screens. In particular embodiments, the synthetic lethality screens can be performed in unmodified strains.

The fluorescent-dye based systems and methods disclosed herein have been demonstrated to be effective in all S. cerevisiae strain backgrounds tested to date, including commonly used lab strains and natural variant strains isolated from different environmental niches. Tested strains including those isolated from oak trees, coffee beans, coconut pods, kefir, sake and Drosophila pseudoobscura (fruit flies). Some tested isolates are tetraploid, triploid or have multiple aneuploidies. Tested lab strains include gene deletions (including a strain that is a heterozygous deletion of an essential gene) and are auxotrophic for multiple amino acids. The method has also been confirmed to be effective in a prototrophic lab strain (no deletions or auxotrophies). Based on the foregoing, systems and methods utilizing fluorescent dyes can reasonably be expected to be effective in any S. cerevisiae strain that sporulates, and any other fungal species that form ascospores, such as Schizosaccharomyces pombe and Neurospora crassa.

Part 2. Capturing the Tetrad Relationship of Recombinant Progeny from a Yeast Cross Using Patterns of Natural Genetic Sequences.

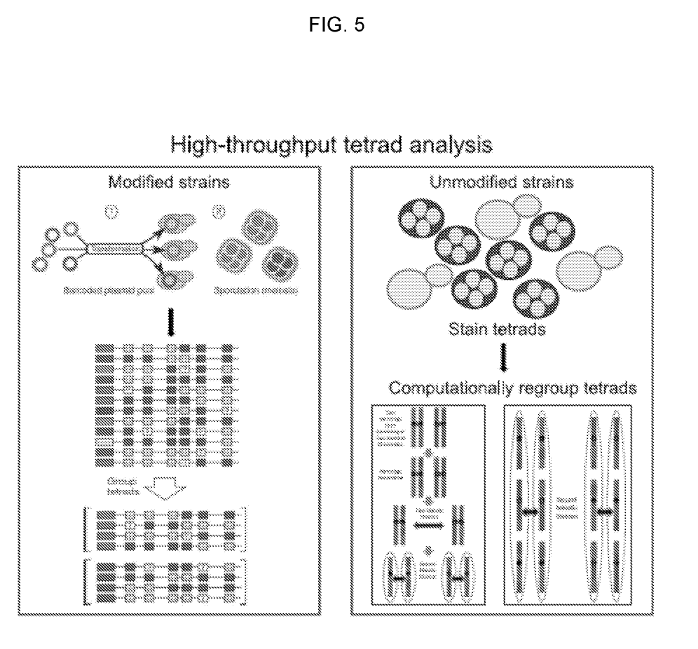

As previously indicated, each spore of a tetrad can give rise to a clonal population of haploid cells, which can be phenotyped and genotyped. Traditional tetrad analysis is a low-throughput process, requiring manual dissection to separate the 4 spores within each tetrad. WO/2014/059370 describes a high-throughput method to replace manual tetrad dissection with fluorescence-activated cell sorting (FACS) of asci onto plates, followed by physical disruption of the tetrads and isolation of individual spores/colonies. The relationship between members of the same tetrad was maintained by the use of a plasmid-located high complexity DNA sequence (“barcode”), specific for each tetrad and inherited by all spores within that tetrad (Ludlow et al., 2013 Nat Methods. 10: 671-675; Scott et al., 2014 J Vis Exp. 87: 51401). Sequencing of this barcode then allowed reconstruction of the tetrad relationships between the recombinant progeny derived from those spores. This approach is depicted in the left panel of FIG. 5 and is contrasted to the currently disclosed methods as depicted in the right panel of FIG. 5.

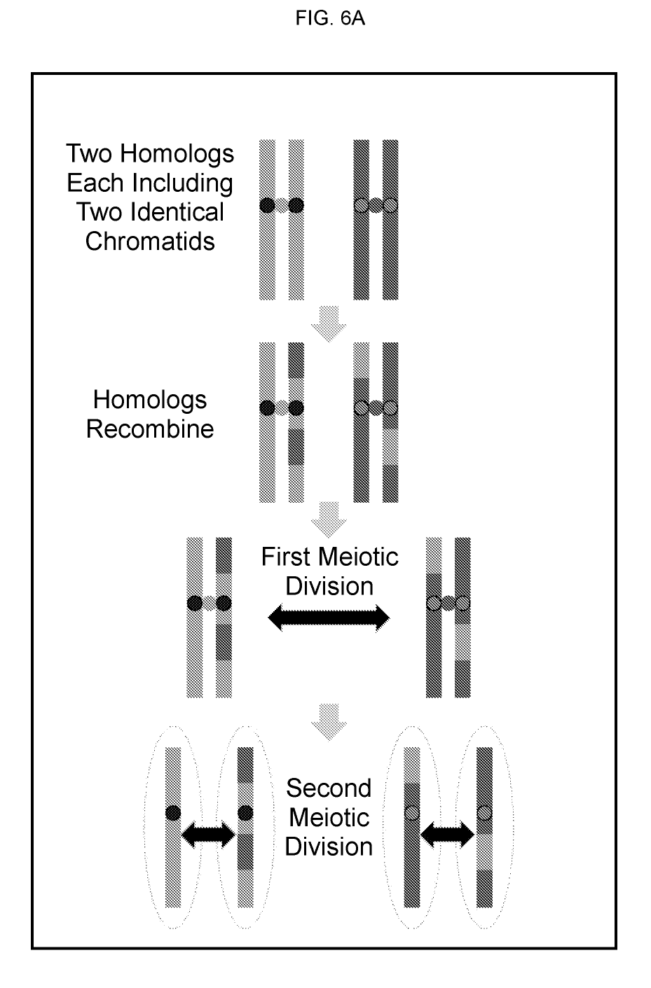

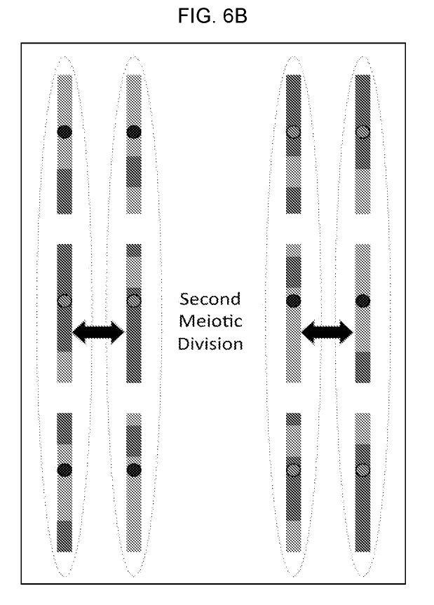

The current disclosure describes reconstruction of tetrad relationships between recombinant progeny by using data obtained from sequencing natural genetic sequences. Natural genetic sequences are DNA sequences encoded by an organism that are not introduced through laboratory-induced genetic manipulation. Meiosis is an example of a naturally-occurring process that alters the genome in ways that can create tetrad-specific markers. Meiosis occurs in diploid cells and produces 4 products, which, in yeast, become the 4 spores of a tetrad. At every position in the diploid that is heterozygous, two spores will inherit the “A” allele and two will inherit the “B” allele (FIG. 6A). In addition, each tetrad is characterized by a unique pattern of relatively sparse recombination events. For example, there are generally 90 crossovers per yeast meiosis, with each spore having 45 crossovers across the entire 12 Mb genome. Therefore, the number of DNA sequence polymorphisms that can be used as genetic markers is much larger than the number of crossovers. Using the information available from these recombination events, it is possible to reconstitute tetrads based only on genome sequencing of the meiotic progeny, dispensing with the tetrad-specific barcode. These methods rely on specific features of tetrads that result from the mechanisms of meiosis. As indicated, in meiosis, a diploid cell undergoes one round of DNA replication followed by two rounds of cell division to produce the four recombinant haploid progeny. In the first meiotic division, the two homologous chromosomes recombine and then segregate to opposite poles of the meiotic spindle. In the second meiotic division, the two chromatids of each recombinant chromosome segregate, essentially as occurs in mitosis (FIG. 6A).

These meiotic processes give rise to the two phenomena that can be utilized in methods disclosed herein. First, at each position heterozygous in the original diploid, in the absence of rare gene-conversion events, exactly two spores will inherit allele “A” and exactly two spores will inherit allele “B” (FIG. 6A). Second, because sister chromatids segregate at the second meiotic division, 2 spores will have matching centromeric alleles for every chromosome, while the other two spores will both have the mirror of this pattern (FIG. 6B).

The obligate 2:2 segregation of heterozygous markers within tetrads (FIG. 6A, 6B) means that among spores from the same tetrad, allele calls are not independent. For example, with knowledge of the genotype of one spore, the allele probabilities in the other three spores change: at every position where the “A” allele is observed in the first spore, the probability of the “A” allele in any of the remaining 3 spores changes from 50% to 33%. Therefore, there are dependencies among the four allele-vectors of a tetrad (one vector for each spore genotype), and also within any group of two or three spores from that tetrad.

These relationships can be detected using information theory methods that utilize only information already contained in the genotypes of the spores without the addition of barcodes or other non-natural genetic modification. These dependencies in the alleles between four spores from the same tetrad mean that mutual information exists between the genome sequences of members of the same tetrad, especially, for example, at the centromeres. Mutual information is a well-known measure that quantifies the amount of dependency between two variables. Interaction information has been proposed (McGill, 1954. Psychometrika. 19, 97-116) as a multivariable generalization of mutual information. Interaction information has a number of advantages and drawbacks (Bell, 2003 “The Co-information Lattice.” In Proc. 4th Int. Symp. Independent Component Analysis and Blind Source Separation 921-926; Jakulin and Bratko, 2004 “Testing the significance of attribute interactions.” In Proc. of the twenty-first international Conf. on Machine learning, 52 pages; Sakhanenko and Galas, 2011 Complexity 17(2): 51-64) but can be used to devise powerful measures of dependency for any number of variables. Interaction information expresses the amount information (redundancy or synergy) bound up in a set of variables, beyond that which is present in any subset of those variables. Unlike mutual information, interaction information can be either positive or negative.

By sequencing enough heterozygous positions genome-wide, information theory techniques can be applied to the genomic data and successfully identify recombinant progeny that arose from the same tetrad and distinguish them from progeny that arose from different tetrads (see, e.g., FIG. 6B). The amount of the genome to sequence depends on the degree of heterozygosity in the diploid parent, with more positions needing to be sequenced when heterozygous sites are uncommon. In test crosses between yeast strains from different populations the methods disclosed herein operate successfully with 3% of the genome sequenced by double digest Restriction-Associated DNA sequencing (RAD-seq). RADseq involves restriction digest of a genome, and sequencing of regions flanking the digested sites. However, any appropriate sequencing or genotyping method can be used. Examples of sequencing methods include light coverage whole genome sequencing by, for example, Illumina, PacBio, or Oxford Nanopore. Light coverage genome sequencing can be defined as genomic sequencing dataset whereby each base in the genome is represented by an average of 5 or fewer reads. Genotyping methods that can be used for tetrad characterization can include high density genotyping by microarray hybridization, RAD-seq, Nanostring hybridization, restriction fragment length polymorphism, and/or polymerase chain reaction (PCR).

In particular embodiments, SNPs between the parental chromosomes in the diploid are used as markers. The number of markers will depend on the degree of heterozygosity between the parental chromosomes and the proportion of the genome sequenced. In previous datasets using diploids derived from strains from different yeast populations and sequencing 3% of the genome marker numbers in the range of hundreds to low thousands were obtained. These markers provided a first step for identifying tetrad relationships.

Searching for tetrads in a large number of spores could be done naïvely by using a brute-force exhaustive search. Before resorting to brute force, however, a heuristic approach based on centromere segregation behavior in tetrads can be used. As discussed above, in a real tetrad, 2 spores have matching alleles at each centromere, while the other two spores will both have the reflected pattern of centromeric alleles (FIG. 6B). Therefore, the heuristic search can be used as a first attempt to partition the set of all spores into clusters of spores whose centromere-flanking markers are either a perfect match or the opposite—a complete mismatch. This can be performed using a greedy algorithm, based on the similarity between spores defined by the edit-distance calculated on the centromere-flanking markers. Delta scores can then be computed within each cluster and tetrads identified.

The relative power of this approach will depend on the number of chromosomes in the organism being analyzed. In the yeast Saccharomyces cerevisiae there are 16 chromosomes so that the probability of either a perfect match, or perfect anti-match between two spores from different tetrads is ½¹⁵. In contrast, the fission yeast S. pombe has only three chromosomes so that the probability of either a perfect match or anti-match is ½². In theory, in S. cerevisiae the heuristic should almost always place spores from the same tetrad in a single cluster. However, due to sequencing errors, crossovers between the centromere-flanking markers and the centromeres, the heuristic can make assignment mistakes. Therefore, the heuristic search alone can be insufficient for high accuracy, but instead can be used to initially reduce the search space. This is also referred to as the divide-and-conquer approach because all spores can first be split into clusters based on the centromere information and the search for tetrads can be performed within each cluster independently. This groups many of the spores into tetrads thus reducing the search space for subsequent analysis.

Reducing the Search Space for Finding Real Tetrads. Based on the foregoing, in particular embodiments, and as disclosed herein, the first grouping step uses a heuristic based on natural genome patterns and can be used to reduce the computational complexity before implementing the second step by subdividing the search space. In other embodiments, such as in organisms like S. pombe with a relatively small number of chromosomes (and thus small number of potential centromere segregation patterns), the second step might be used more heavily or even exclusively.

Particular embodiments of the disclosed methods start with a set of spores representing all of the members of a group of tetrads, but with tetrad identity lost, such as in the high-throughput tetrad isolation and disruption method BEST (Ludlow et al., 2013 Nat Methods. 10: 671-675). These spores are grown into individual colonies from which DNA is isolated followed by, for example, whole-genome sequencing or high-density genome-wide genotyping, for example using RAD-seq (WO 2006/122215; U.S. Pat. No. 9,365,893) (Baird et al., PLoS One 3: e3376, 2008). In particular embodiments, a two-step informatics approach can then be used two organize these recombinant progeny into their original tetrad relationships. In a first step, spores are grouped into potential tetrads based on their redundant and mirrored features in the natural genome (e.g., redundant and mirrored centromere-linked markers), while in a second step any such groupings that include multiple tetrads are refined down into single tetrads.

In particular embodiments, irst, redundant and mirrored features in the natural genome are used to group colonies into potential tetrads. This example describes the use of redundant and mirrored centromere-linked markers. Meiosis includes two divisions with recombinant homologous chromosomes separating in the first division, and sister chromatids in the second (FIG. 6A). Each of the two products of the first meiotic division gives rise to two spores and each of these pairs have matching alleles at each of their centromeres (they are recombinant in their arms) (FIG. 6B). Here, there should always be a complete match unless recombination has occurred between the marker and the centromere, there has been a genotyping error, or meiotic chromosome missegregation has occurred. In these embodiments, recombinant progeny are only considered for grouping when they lack crossovers between the 2 markers flanking the centromere on every chromosome. Grouping is done with no error allowed, spores discarded at this step have a chance to be grouped into tetrads during the one or more second steps described below.

In budding yeast, the centromeres are short sequences (120 bp). In particular embodiments, the centromere allele is defined based on the alleles observed at the closest flanking markers. This is done only when those markers have the same allele (both “A” or both “B”), i.e. no recombination detected in the centromere interval. The methods disclosed herein can be agnostic as to centromere length, but do require that the flanking markers display strong genetic linkage to the centromere. Note that in particular embodiments, incorrectly grouped recombinant progeny will not be placed into tetrads because they will fail to pass the second step, described below. These spores can be grouped into tetrads at later steps.

In particular embodiments, the cut-offs to define a match between two spores using only centromere flanking markers are: at least 50% shared valid markers flanking centromeres, and perfect consensus between these markers. Valid means not missing, and no transition from one parent to another (which might indicate a crossover near the centromere).

In addition to utilizing centromere markers, and again referring to FIGS. 6A and 6B, recombinant progeny relationships can also be identified based on allele frequencies and/or cross-over patterns. These patterns are created by the presence of four copies of each chromosome (two from one parent and two from the other parent) reciprocal nature of crossing over during meiotic recombination. As depicted schematically in FIGS. 6A and 6B, for a spore from a given tetrad with a given recombination event, another spore from that same tetrad will harbor a reciprocal recombination event. As such, the 2:2 allele ratio of a given alleles are maintained between the four spores of a true tetrad, but not between four randomly selected spore from different tetrads or even three spore from the same tetrad and one spore assigned to that tetrad in error. Thus, the patterns of the recombination events themselves or the allele ratios that they generate can be used to identify recombinant progeny that arose from the same meiotic event (sister spores within the same tetrad).

When applied to large hand-dissected datasets, organizing spores by these methods has been 100% successful in placing members of the same tetrad in the same allele-group. To further support this disclosure, the probability of 2 spores from different tetrads having the same centromere pattern is ½¹⁶ i.e. 1/65536. With 500 spores there are 124750 pairwise spore comparisons, but with 100 spores there are only 4950, so this problem will depend very much on the total number of spores analyzed. As expected, this effect is seen in datasets having several hundred strains. In a smaller set of 432 spores, there were 8 instances, and in a larger dataset set of 1143 spores, there were 22 instances where looking at markers near the centromeres did not provide enough information for re-grouping. If spores in the dataset contain missing markers which flank centromeres, this will increase the ambiguity and the need for a second technique to group spores into tetrads.

As indicated, in particular embodiments, a second step is additionally or alternatively used to group spores into their original tetrad relationships. Particular embodiments can utilize mutual information, clustering algorithms (e.g. k-means clustering where k=the number of tetrads expected based on the number sorted), Markov chains, or even simple pattern matching looking for reciprocal recombination events. A Markov chain is a type of Markov process that has either discrete state space or discrete index set.

Particular embodiments can utilize the second method based on information theory (Sakhanenko & Galas, 2015. J. Comp. Biol. 22(11):1005-1024) to organize recombinant progeny into tetrads, starting with the groupings identified by the redundant and mirrored features in the natural genome approach. In particular embodiments, this second step can be necessary because, with sufficiently large numbers of progeny to analyze, and particularly if genotyping information is missing for some centromeres, multiple tetrads may share a genome sequence pattern. In particular embodiments, informatics methods consider all markers genome-wide and calculate an information-theory-based score for each pair, triple and quad of progeny within each grouped set as well as within a set of progeny with an ambiguous relationship. High scores correspond to progeny that share common information and thus are more likely to have originated from the same tetrad. Score cutoffs derived from the whole dataset can then be used to identify the true tetrad-groupings. In particular embodiments, the random background distribution of scores can be constructed, and a cutoff is selected to be significantly distinct from the background distribution.

The pair-wise score is mutual information between progeny derived from two spores. Mutual information measures how much information one variable carries about the other variable. For tuples with N spores (N>2), the score corresponds to conditional interaction information, which is the expected value of the interaction information for N−1 variables given the value of the Nth variable. Since conditional interaction information is asymmetric relative to the conditional variable, the product of conditional interaction information across all conditional variables is taken.

Referring and quoting passages from Sakhanenko and Galas more particularly, in particular embodiments the following approaches can be used:

Interaction information for three-variable dependency is described. The three-variable interaction information, I(X₁, X₂, Y), can be thought of as being based on two predictor variables, X₁ and X₂, and a target variable, Y. The three-variable interaction information can be written as the difference between the two-variable interaction information, with and without knowledge of the third variable:

I(X₁,X₂,Y)=I(X₁,X₂|Y)−I(X₁,X₂), (1)

where I(X₁, X₂) is the mutual information, and I(X₁, X₂|Y) is conditional mutual information given Y. When expressed entirely in terms of marginal entropies:

I(X₁,X₂,H(X₁)+H(X₂)+H(Y)−H(X₁,X₂)−H(X₁,Y)−H(X₂,Y)+H(X₁,X₂,Y) (2)

H(Xᵢ) is entropy of a random variable Xᵢ, and

H(Xₖ_{₁}, . . . ,Xₖ_{ₘ}),m≥2,

is a joint entropy on a set of m random variables.
Considering the interaction information for multiple variables for a set of n variables,

νₙ={X₁,X₂, . . . ,X₂},

the interaction information can be written in terms of sums of marginal entropies according to the inclusion-exclusion formula, which is the sum of the joint entropies of νₙ:

$$I ( {v}_{n} ) = - {\sum }_{τ ⊆ {v}_{n}} {( - 1 )}^{\vert τ \vert} H ( τ ) \qquad ( 3 )$$

Given Equation 3, the “differential interaction information,” Δ, is defined as the difference between values of successive interaction informations arising from adding variables:

Δ(X₁,νₙ)=[I(νₙ)−I(νₙ\{Xᵢ})]=−I(ν_{ν}\{Xᵢ}|Xᵢ) (4)

The last equality comes from the recursive relation for the interaction information, Equation 1. The differential interaction information is that change in interaction information that occurs when another variable is added to the set of n−1 variables. This differential can then be written using the marginal entropies. If {τᵢ} are all the subsets of νₙ that contain Xᵢ (note: this is not all subsets) then:

$$Δ ( {X}_{i} , {v}_{n} ) = {\sum }_{\{ {τ}_{i} ⊆ {v}_{n} \vert {X}_{i} ∈ {τ}_{i} \}} {( - 1 )}^{\vert {τ}_{i} \vert + 1} H ( {τ}_{i} ) \qquad ( 5 )$$

Then Δ's for degrees (the number of variables) three and four (denoting the corresponding variables in the subscripts) are:

Δ(X₂,ν₃)=I₁₂₃−I₁₃=H₂−H₁₂−H₂₃+H₁₂₃

Δ(X₁,ν₃)=I₁₂₃−I₂₃=H₁−H₁₂−H₁₃+H₁₂₃

Δ(X₁,ν₄)=I₁₂₃₄−I₂₃₄=H₁−H₁₂−H₁₃−H₁₄+H₁₂₃+H₁₂₄+H₁₃₄−H₁₂₃₄ (6)

The number of terms grows as the power of the number of variables minus one. For the case when the variables are all independent, all elements of Δ(Xᵢ, νⱼ) in Equation 4 are zero. These expressions are zero for all numbers of variables, as the joint marginal entropies become additive single entropies and all terms cancel.

The differential interaction information in Equation 4 is based on specifying the target variable, the variable added to the set of n−1 variables. The differential is the change that results from this addition and is therefore asymmetric in that variable designation (and thus not invariant under permutation.) See Equation 6 for an example of using different target variables. Since the purpose is to detect fully cooperative dependence among the variable set, any single measure should be symmetric. A more general measure then can be created by a simple construct that restores symmetry. If Δ's are multiplied with all possible choices of the target variable the resulting measure will be symmetric and will provide a general measure that is functional and straightforward. To be specific, the symmetric measure is defined as

$${\overline{Δ}}_{n} = {\overline{Δ}}_{n} ( {v}_{n} ) ≡ {( - 1 )}^{n} {\prod }_{i = 1}^{n} [ I ( {v}_{n} ) - I ( {v}_{n} \backslash \{ {X}_{i} \} ) ] , \qquad ( 7 )$$

where the product is over the choice, i, of a target variable relative to νₙ, n>2, a simple permutation. The difference terms in the bracket in Equation 7 are between the interaction information for the full set νₙ (first term) minus the interaction information for the same set minus a single element. For three variables this expression is (simplifying the notation again)

Δ₃(X₁,X₂,X₃)=(−1)³×(H₁−H₁₂−H₁₃+H₁₂₃)×(H₂−H₁₂−H₂₃+H₁₂₃)×(H₃−H₁₃−H₂₃+H₁₂₃) (8)

This measure has the extremely useful property that it is always small or vanishes unless all variables in the set are interdependent. This can be used to allow discovery and representation of exact variable dependencies.

In particular embodiments, an exhaustive search is used to group the remaining spores, when possible, into tetrads. The exhaustive search is more computationally expensive than the divide-and-conquer approach (first step) so combining these two techniques in this order results in a more computationally efficient (i.e. quicker) analysis that simply using the exhaustive search on the entire search space.

As indicated, analysis of complex biological systems measures are needed that can detect synergistic, multiple variable dependencies. Mutual information is a well-known measure that quantifies the amount of dependency between two variables:

$$I ( X , Y ) = H ( X ) + H ( Y ) - H ( X , Y ) = {\sum }_{x ∈ X , y ∈ Y} p ( x , y ) log \frac{p ( x , y )}{p ( x ) p ( y )} \qquad ( 9 )$$

The interaction information for three variables, for example, quantifies the difference between the two-variable interaction information (mutual information), with and without knowledge of the third variable:

$$I ( X , Y , Z ) = I ( X , Y ) - I ( X , Y \vert Z ) = H ( X ) + H ( Y ) + H ( Z ) - H ( X , Y ) - H ( X , Z ) - H ( Y , Z ) + H ( X , Y , Z ) \qquad ( 10 )$$

Here I(X, Y|Z) is conditional mutual information, H(X) is entropy of variable X and H(X, Y, Z) is a joint entropy of the three variables. Note that the conditional mutual information is actually a difference between interaction informations for two and three variables—a differential interaction information. A general form of interaction information for the set of νₙ variables, in terms of marginal entropies can be written as:

$$I ( {v}_{n} ) = - {\sum }_{τ ⊆ {v}_{n}} {( - 1 )}^{\vert τ \vert} H ( τ ) \qquad ( 11 )$$

In these embodiments, a symmetric product of differential interaction information called “delta” (Galas et al., 2014. J. Comp. Biol. 21(2), 118-140; Sakhanenko & Galas, 2015. J. Comp. Biol. 22(11):1005-1024; Galas & Sakhanenko, 2016. Multivariate information measures: a unification using Möbius operators on subset lattices. arXiv1601.06780) can be used. Differential interaction information quantifies the change in interaction information that occurs when another variable is added to a set of variables, so for three variables it is defined as:

$$Δ ( \{ X , Y \} ; Z ) = - I ( X , Y \vert Z ) = I ( X , Y , Z ) - I ( X , Y ) = H ( Z ) - H ( X , Z ) - H ( Y , Z ) + H ( X , Y , Z ) \qquad ( 12 )$$

If νn={X₁, X₂ . . . Xₙ} and νi=νn−{Xₙ} then the differential interaction information can be defined in general as

$$Δ ( {v}_{i} ; {X}_{i} ) = I ( {v}_{i} ) - I ( V ) = {\sum }_{{τ}_{i} ⊆ V \vert {X}_{i} ∈ {τ}_{i}} {( - 1 )}^{\vert {τ}_{i} \vert + 1} H ( {τ}_{i} ) \qquad ( 13 )$$

Note that, unlike interaction information, differential interaction information is not symmetric, because X in equation 5 is a special variable. In order to create a symmetric measure, the product of differential interaction information is taken with all possible choices of the target variable:

$${\overline{Δ}}_{m} = {\prod }_{i = 1}^{m} Δ ( {v}_{i} ; {X}_{i} ) \qquad ( 14 )$$

Δ̅ₘ is referred to as the delta measure, for m variables. Although this is a general, multivariable measure, in these embodiments the focus is on delta computed only on 3- and 4-variable sets. Three- and 4-variable delta, as well as the pair-wise measure, mutual information can be used to scan the data from large sets of yeast spores and detect and assemble spore tetrads and their components.

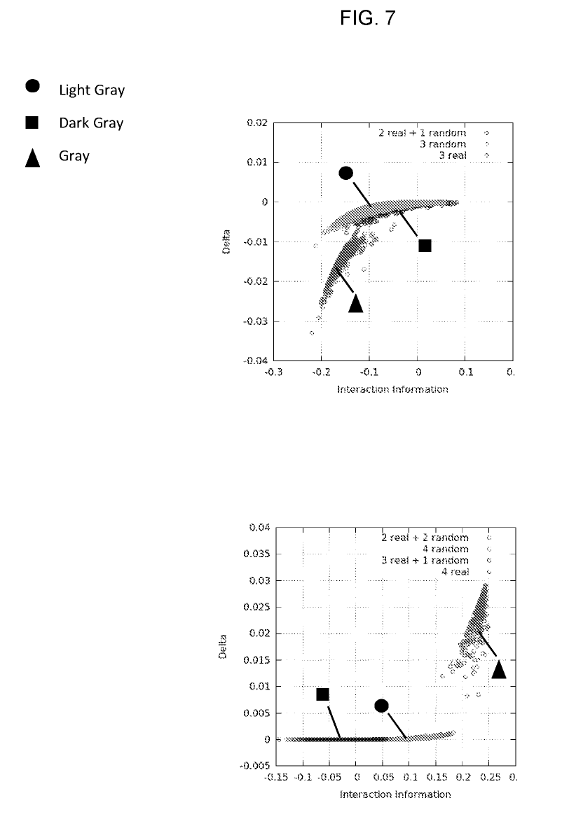

Simulated Data Validation. Utilizing the approach described in relation to Equations 9-14, FIG. 7 compares delta with interaction information for 3 spore (top panel) and 4 spore (bottom panel) cases. A simulated data set with 1461 markers and 1140 spores from 285 tetrads was used. When interaction information was applied to the genotypes of spores from this simulated spore dataset, groups of 3 and 4 spores from real tetrads scored strongly, as expected. A “real” tetrad is a set of genomic data from four spores that are known (because the data is simulated) to have originated from the same tetrad. However, while most incorrect groups of spores scored poorly, some scored as highly as some of the correct groups (FIG. 7) i.e. there is noise in these null distributions, particularly at the 3-spore level.

In particular embodiments, to combine the interaction information at different spore-number levels, the delta measure (Galas et al., 2014. J. Comp. Biol. 21(2), 118-140; Sakhanenko & Galas, 2015. J. Comp. Biol. 22(11):1005-1024; Galas & Sakhanenko, 2016. Multivariate information measures: a unification using Möbius operators on subset lattices. arXiv1601.06780), which is based on differential interaction information can be used. Differential interaction information quantifies the change in interaction information that occurs when another variable is added to a set of variables. Note that, unlike interaction information, differential interaction information is not symmetric, but is specific to which target variable is considered “added.” In order to create a symmetric measure, the product of differential interaction information is taken with all possible choices of the target variable.

Delta performed better than interaction information in distinguishing real tetrads from incorrect groups of spores (FIG. 7). It can be seen that interaction information scores are high for groups of 3 or 4 spores coming from the same tetrad. However, as discussed above, these scores overlap considerably with the scores computed on groups of spores from different tetrads even in the 4-spore case where the real-tetrad signal is strongest. By combining the information at the 3- and 4-spore levels, the delta measure allows distinction of real tetrads from all other 4-spore groups. And even in the 3-spore case, the delta measure considerably reduces the ambiguity between 3-spore groups that come from the same tetrad from the groups constructed from different tetrads (FIG. 7, top panel).

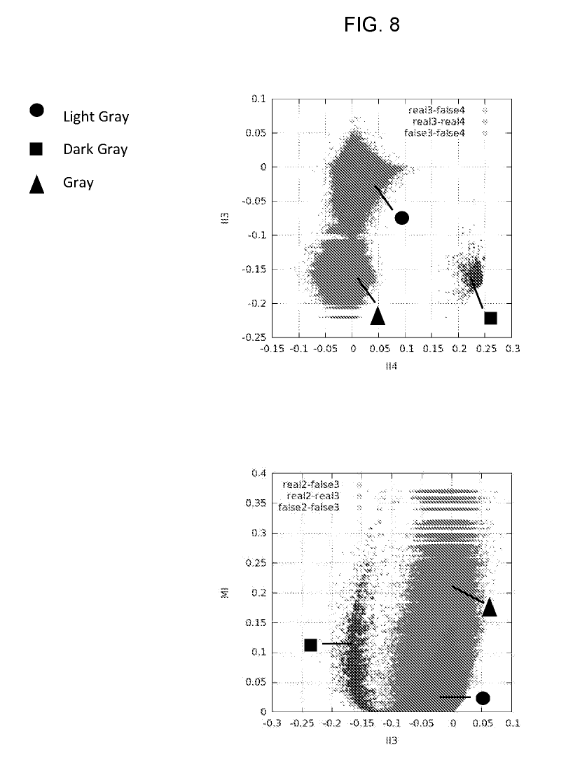

FIG. 8 shows an example of combining information at the 3- and 4-spore level (top panel) and the 3- and 2-spore level. Importantly, the high interaction information associated with real tetrads was observed at both the 4-spore level and also at the 3-spore level for all subgroups of 3 spores from that tetrad (FIG. 8, top panel). In contrast, while an incorrect tetrad might score highly at the 4-spore level, that did not extend to the 3-spore level for its subgroups, i.e. the noise at the 3- and 4-spore levels is not correlated (FIG. 8, top panel). Therefore, if the interaction information at the 4-spore level is combined with that from the 3-spore level, the signal separating real 4-spore tetrads from false ones should be much stronger than using interaction information alone (FIG. 8, top panel showing a dearly defined cluster in the lower right). A similar pattern of uncorrelated noise was seen for the 2- and 3-spore levels, although the noise level was higher (FIG. 8, bottom panel) and so combining interaction information at the 3- and 2-spore levels should also increase the ability to identify real tetrads with only 3 viable spores.

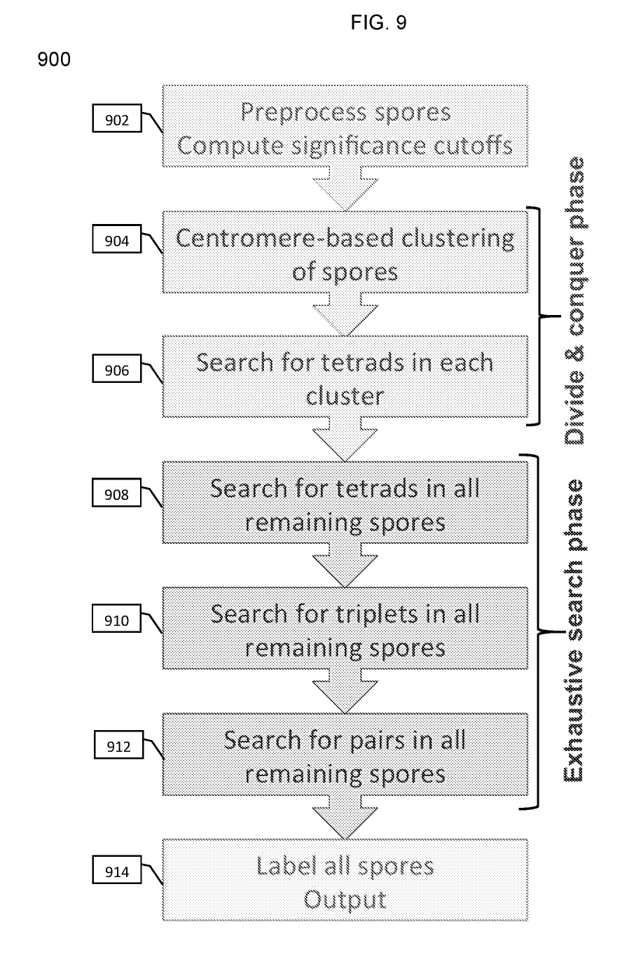

Exemplary Methods. FIGS. 9-11 show aspects of exemplary methods of identifying tetrad relationships based on genomic data obtained from individual spores. For ease of understanding, the methods discussed in this disclosure are delineated as separate operations represented as independent blocks. However, these separately delineated operations should not be construed as necessarily order dependent in their performance. The order in which the process is described is not intended to be construed as a limitation, and any number of the described process blocks may be combined in any order to implement the methods, or alternate methods. Moreover, it is also possible that one or more of the provided operations may be modified or omitted.

FIG. 9 shows an exemplary method 900 for computationally inferring tetrad relationships from randomly arrayed yeast spores. Method 900 includes four phases: a data preprocessing and set up phase (block 902), a divide-and-conquer heuristic search phase (blocks 904 and 906), an exhaustive search phase (blocks 908-912), and an output phase (block 914).

At block 902, the data is preprocessed to remove strains with low numbers of marker calls and highly “heterozygous” strains, likely reflecting contamination of one strain by another.

At block 904, spores are next clustered using the centromere heuristic.

At block 906, tetrads are identified within these clusters based on exhaustive searches using delta, first searching for 4-spore tetrads, and then 3-spore.

At block 908, an exhaustive comparison of all spores unassigned to a tetrad is undertaken using delta, first for 4-spore, then for 3-spore at block 910 and, finally, for 2-spore tetrads at block 912. This completes the assembly of the spores into tetrads.

At block 914, the output being the tetrad labels for each of the spores is generated.

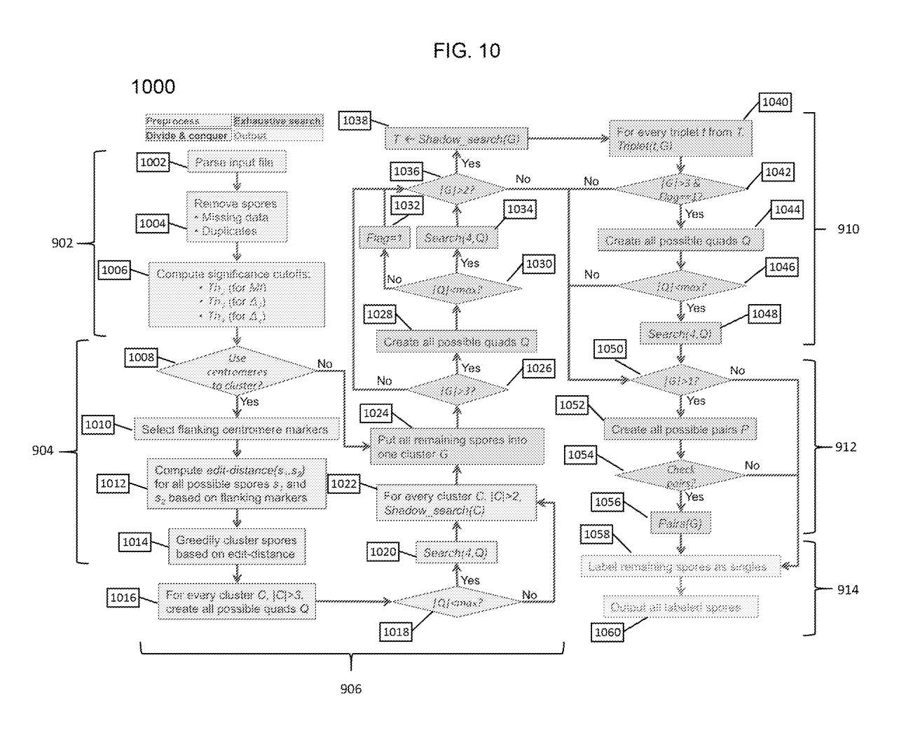

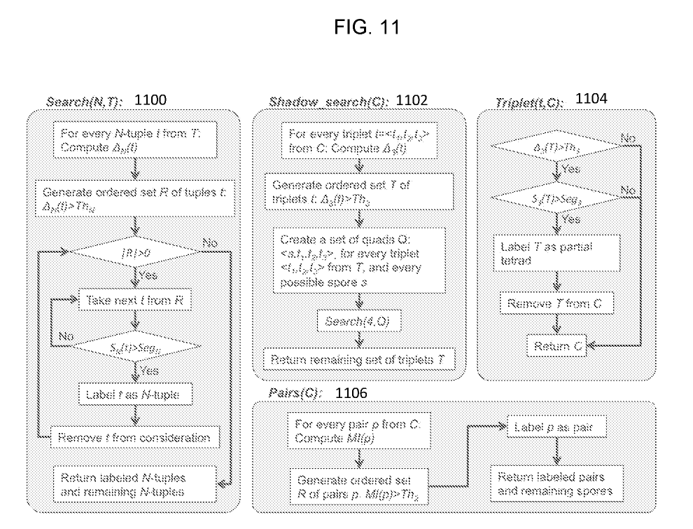

FIG. 10 shows exemplary method 1000, which provides additional details for each of the four phases introduced in FIG. 9.

The preprocessing portion of method 1000 corresponds to block 902 of FIG. 9. At block 1002, an input file is parsed. The input file may contain the genomic information from the spores. In particular embodiments, the input file may be in any format suitable for representing genomic information as electric data such as a text file, a FASTA file, or other file type. The input file can be received directly from a DNA sequencer or obtained indirectly via a network, memory device, or other computing device.

At block 1004, the preprocessing continues by removing spores, identifying missing data, and removing duplicate entries.

At block 1006, cutoff thresholds are computed. In particular embodiments, the thresholds are used to identify the candidate tetrads of spores (as well as triplets and pairs—incomplete tetrads).

The next phase, the heuristic search phase, can begin at block 1008. At block 1008, it is determined if the method will use centromeres to cluster spores into tetrads. If this technique is not used, method 1000 proceeds along the no path to block 1024. This first grouping technique using centromeres may be skipped for organisms like S. pombe with a relatively small number of chromosomes. If, however, centromeres are used to cluster then method 1000 proceeds along the yes path to block 1010.

At block 1010, flanking centromere markers are selected. The information contained in these markers is used to cluster the spores.

At block 1012, edit-distances are computed for all possible spore pairs based on the flanking markers. Edit distance is a way of quantifying how dissimilar two strings (e.g. words) are to one another by counting the minimum number of operations required to transform one string into the other. In bioinformatics, edit distance can be used to quantify the similarity of DNA sequences, which can be viewed as strings of the letters A, C, G and T. In particular embodiments, if two spores are close to each other (which is user-defined) according to the edit-distance computed on the flanking markers, then these two spores are assigned to the same cluster.

At block 1014, spores are formed into clusters based on edit-distance using a clustering algorithm. This attempts to partition the set of all spores into clusters of spores whose centromere-flanking markers are either a perfect match or the opposite—a complete mismatch. One suitable type of clustering algorithm is a greedy algorithm. Blocks 1008-1014 correspond to block 904 of FIG. 9.

At block 1016, for every cluster C in which there are four or more spores, method 1000 attempts to create all possible groupings of four spores called “quads” Q. In particular embodiments, a quad is not necessarily a tetrad.

At block 1018, it is determined if the number of quads is less than a maximum number. The maximum number may be user defined and may be based on the processing power of a computing device implementing method 1000. If the number of quads is less than the maximum number, then process 1000 proceeds along the yes path to 1020.

At block 1020, an exhaustive search is performed for all tetrads on all the spores in each cluster. Identified tetrads are not included in subsequent analysis. In particular embodiments, if the number of quads is such that performing an exhaustive search would be too computationally expensive, then method 1000 proceeds from block 1018 along the no path to 1022.

At block 1022, for all spores remaining in a cluster of two or more spores that were not included in a tetrad, a “shadow search” is performed. Details of the shadow search are described below in the discussion of FIG. 11. Blocks 1016-1022 correspond to block 906 of FIG. 9.

The third phase, the exhaustive search phase, begins at block 1024. At block 1024, all remaining spores that are not part of a tetrad are grouped into one cluster G. In particular embodiments, if the set of all remaining spores is not too large, the first search is for tetrads exhaustively.

At block 1024, in particular embodiments it is determined if there are more than three spores in cluster G. If not, method 1000 proceeds to triplet analysis at block 1036. If yes, then method 1000 follows the yes path to block 1028.

At block 1028, all possible quads are created from the spores in cluster G. This is similar to block 1016.

At block 1030, it is determined if the number of quads is less than the maximum number. This is similar to block 1018. In particular embodiments, if the number is equal or greater than the maximum number, method 1000 proceeds to block 1032 and flags the remaining spores for later analysis. If the number of quads is less than the maximum, then method 1000 proceeds to block 1034.

At block 1034, the exhaustive search is performed. This is similar to block 1020. Any complete tetrads are identified and those spores are excluded from further analysis. Blocks 1024-1034 correspond to block 908 (search for tetrads) of FIG. 9.

At block 1036, in particular embodiments it is determined if there are more than two spores in the cluster G. If not, method 1000 proceeds along the no path to block 1050 and begins pair analysis. If there are at least three spores, method 1000 proceeds along the yes path to block 1038.

At block 1038, a shadow search is performed to find and remove spores that form complete or incomplete tetrads (triplets). The shadow search identifies triplets that can form tetrads by the addition of an additional spore. The tetrads are removed from the set of triplets T.

At block 1040, in particular embodiments every triplet in T is identified as a partial tetrad.

At block 1042, in particular embodiments it is determined if there are more than three spores remaining in cluster G and spores flagged at block 1032 are also analyzed. If there are fewer than three spores, then method 1000 proceeds along the no path to block 1050 and performs pair analysis. If there are more than three spores, quad formation is possible, and process 1000 proceeds along the yes path to 1044.

At block 1044, all possible quads are created from the triplets and single spores in cluster G. This is similar to blocks 1016 and 1028.

At block 1046, it is determined if the number of quads is less than the maximum number. This is similar to blocks 1018 and 1030. If the number is equal or greater than the maximum number, method 1000 proceeds to block 1050 and begins pair analysis. If the number of quads is less than the maximum, then method 1000 proceeds to block 1048.

At block 1048, the exhaustive search is performed. This is similar to blocks 1020 and 1034. Any complete tetrads are identified and those spores are excluded from further analysis. Blocks 1036-1048 correspond to block 910 (search for triplets) of FIG. 9.

At block 1050, in particular embodiments it is determined if there is more than one spore remaining in cluster G. If not, then method 1000 proceeds along the no path to block 1058. If there are two or more spores, then method 1000 proceeds along the yes path to block 1052.

At block 1052, in particular embodiments all possible pairs are created.

At block 1054, the possible pairs are checked to see if they form an incomplete tetrad. For spores that do form a pair with another spore, method 1000 proceeds along the yes path to block 1056. For single spores that do not form a pair with any other spore, method 1000 proceeds along the no path to block 1058.

At block 1056, in particular embodiments all pairs in cluster G are identified as partial tetrads. Thus, at this stage in method 1000, all possible tetrads and partial tetrads (triplets and pairs) have been formed from the spores in cluster G. Blocks 1050-1056 correspond to block 912 (search for pairs) of FIG. 9.

The fourth phase of output begins at block 1058. At block 1058, in particular embodiments all remaining spores that have not been included in a tetrad, triple, or pair are labeled as singles.

At block 1060, all labeled spores (tetrads, triples, pairs, and singles) are output. The output may include generating data in a human-usable form such as outputting information onto a display of a computing device, printing the output data, etc. Blocks 1058 and 1060 correspond to block 914 of FIG. 9.

FIG. 11 shows methods corresponding to specific functions used in method 1000 and shown in FIG. 10. The specific functions are a search function 1100 for N-tuples in a set of tuples T, a shadow search function 1102 for tetrads and triplets in cluster C, a triplet function 1104 for identification of triplets tin cluster C, and a pairs function 1106 for identification of pairs in cluster C.

The search function 1100 encodes the exhaustive search for N-tuples (tetrads if N=4) in the set of tuples T. In particular embodiments, delta scores for all tuples in the set are computed (Δ_{N}(t)), and those tuples that pass the significance filter (above the threshold) are tested for the 2-2 segregation (or 2-1 segregation in case of triplets) and successfully labeled and removed from further consideration. The search function 1100 returns all labeled N-tuples as well as the set of remaining N-tuples. This search function 1100 is used to perform an exhaustive search in blocks 1020, 1034, and 1048 of FIG. 10.

The shadow search function 1102 encodes a search based on the heuristic that many tetrads contain triplets of spores with significant delta scores. In particular embodiments, the shadow search function 1102 computes the delta scores for all triplets of spores ((Δ₃(t)) from the cluster C. For those triplets that pass the significance filter, the function creates a set of 4-tuples by combining the triplets with all other spores and performs the exhaustive search (i.e. Search(4,Q)). The shadow search function 1102 then removes successfully identified tetrads and returns the set of remaining triplets T that passed the significance filter. The shadow search function 1102 is used in FIG. 10 at blocks 1022 and 1028.

The triplet function 1104 identifies triplets based on delta scores. In particular embodiments, the triplet function 1104 computes the delta score for the triplet t, checks that it passes the significance filter and the 2-1 segregation filter, labels it as an incomplete or partial tetrad and removes from the cluster C. The triplet function 1104 is included in FIG. 10 at block 1040.

The pairs function 1106 identifies pairs of spores based on mutual information (MI) scores. In particular embodiments, the pairs function 1106 computes mutual information scores for all pairs in the cluster C and labels those pairs that pass the significance filter as incomplete tetrads (i.e. as pairs). Any spores remaining are returned as single spores. The pairs function 1106 is included in FIG. 10 at block 1052.

The described methods successfully identify tetrad relationships utilizing natural genetic sequences.

Part 3. Detection of Genetic Recombination Events.

This aspect of the disclosure describes methods to detect genetic recombination events in genomic regions of interest. The embodiments utilize markers in the genomes of an offspring's parents.

In particular embodiments, a genetic construct encoding a marker of a marker pair is inserted into one parent's genome and a second genetic construct encoding the second marker of the marker pair is inserted into the second parent's genome. If both markers of the pair are expressed together in the offspring, a detectable or differential signal distinct from the signal of either member of the pair alone is generated, thus identifying a genetic recombination event in the genomic region of interest. An exemplary marker pair includes two different drug resistance markers or two different fluorescent proteins.

In particular embodiments, a genetic construct encoding one element of a split marker pair is inserted into one parent's genome and a second genetic construct encoding a second element of the split marker pair is inserted into the second parent's genome. If the split marker's components are expressed together in the offspring, a detectable or differential signal distinct from the signal of either half of the split marker alone is generated, thus identifying a genetic recombination event in the genomic region of interest.

In particular embodiments, drug resistance markers can be utilized as markers. Exemplary drug resistance markers include acetamide assimilation genes (Kelly & Hynes, EMBO J. 4: 475-479, 1985); benomyl resistance genes (Koenraadt, et al. 1992); bialaphos resistance genes (Avalos et al., Curr. Genet. 16: 369-372, 1989); bleomycin t (pleomycin) resistance genes (Punt et al., Meth Enzymol. 216: 447-457, 1992); hygromycin (Hygromycin B) resistance genes; and sulfonylurea resistance genes (Zhang et al., Appl Microbiol Biotechnol. 87: 1151-1156, 2010).

In particular embodiments, fluorescent proteins and analogs thereof can be utilized as markers. Exemplary fluorescent proteins include blue fluorescent proteins (e.g. eBFP, eBFP2, Azurite, mKalamal, GFPuv, Sapphire, T-sapphire); cyan fluorescent proteins (e.g. eCFP, Cerulean, CyPet, AmCyanl, Midoriishi-Cyan); green fluorescent proteins (e.g. GFP, GFP-2, tagGFP, turboGFP, eGFP, Emerald, Azami Green, Monomeric Azami Green, CopGFP, AceGFP, ZsGreenI); orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRFP1, DsRed-Express, DsRed2, DsRed-Monomer, HcRed-Tandem, HcRedI, AsRed2, eqFP611, mRaspberry, mStrawberry, Jred); yellow fluorescent proteins (e.g., YFP, eYFP, Citrine, Venus, YPet, PhiYFP, ZsYellowI); and any other suitable fluorescent proteins known to those of ordinary skill in the art, including firefly luciferase. Specific, non-limiting examples of split fluorescent proteins include those described in Paulmurugan et al. (PNAS USA 99(24):15608-15613, 2002) and Demidov et al. (PNAS USA 103(7):2052-2056, 2006). See also International Patent Publication No. WO 2012/135535; U.S. Patent Publications 2012-0282643, 2015-0099271, 2015-0010932, and 2014-0024555; and U.S. Pat. Nos. 8,685,667 and 9,081,014.

Particular embodiments can also utilize cerulenin resistance genes (e.g., fas2m, PDR4; Inokoshi et al., Biochemistry 64: 660, 1992; Hussain et al., Gene 101: 149, 1991); copper resistance genes (CUP1; Marin et al., Proc. Natl. Acad. Sci. USA. 81: 337, 1984); and geneticin resistance gene (G418r) as markers.

Additional useful markers include β-galactosidase (β-gal) and β-glucuronidase (GUS) (see, e.g., European Patent Publication EP2423316). These reporter proteins function by hydrolyzing a secondary marker molecule (e.g., a β-galactoside or a β-glucuronide). Thus it will be understood that methods and systems that employ one of these marker proteins will also involve providing the compound(s) needed to produce a detectable reaction product. Assays for detecting β-gal or GUS activity are well known in the art.

In some embodiments it may be appropriate to use auxotrophic markers as markers. Exemplary auxotrophic markers include methionine auxotrophic markers (e.g., met1, met2, met3, met4, met5, met6, met7, met8, met10, met13, met14 or met20); tyrosine auxotrophic markers (e.g., tyr1 or isoleucine); valine auxotrophic markers (e.g., ilv1, ilv2, ilv3 or ilv5); phenylalanine auxotrophic markers (e.g., pha2); glutamic acid auxotrophic markers (e.g., glu3); threonine auxotrophic markers (e.g., thr1 or thr4); aspartic acid auxotrophic markers (e.g., asp1 or asp5); serine auxotrophic markers (e.g., ser1 or ser2); arginine auxotrophic markers (e.g., arg1, arg3, arg4, arg5, arg8, arg9, arg80, arg81, arg82 or arg84); uracil auxotrophic markers (e.g., ura1, ura2, ura3, ura4, ura5 or ura6); adenine auxotrophic markers (e.g., ade1, ade2, ade3, ade4, ade5, ade6, ade8, ade9, ade12 or ade15); lysine auxotrophic markers (e.g., lys1, lys2, lys4, lys5, lys7, lys9, lys11, lys13 or lys14); tryptophan auxotrophic markers (e.g., trp1, trp2, trp3, trp4 or trp5); leucine auxotrophic markers (e.g., leu1, leu2, leu3, leu4 or leu5); and histidine auxotrophic markers (e.g., his1, his2, his3, his4, his5, his6, his7 or his8).

In particular embodiments, the genetic constructs include regulatory sequences to control the expression of the nucleic acid molecules. In particular embodiments, the regulatory sequence can result in the constitutive or inducible expression of markers encoded by the genetic construct.

In particular embodiments, the regulatory sequences to control expression of the genetic constructs include promoters selected for use to enable autonomous expression in spores. Exemplary promoters include Saccharomyces promoters such as pADH1, pTDH3, pPGK1, pADH2, pPDC2, pPMA1 and pGPD1.

In particular embodiments, the regulatory sequences can include or encode an interaction domain, for example, to drive sufficient refolding of a split marker protein to allow for function and signal creation. Exemplary interaction domains include protein-protein interaction domains such as EF1, EF2, SH2, SH3, PDZ, 14-3-3, WW and PTB and Notch and Delta ectodomains, as well as integrin α and β subunits.

In particular embodiments, the regulatory sequences can include or encode a restriction site. Following recombination events, restriction sites can flank the genomic region of interest such that when genomic DNA is digested with the appropriate enzyme, a fragment that can be isolated by size selection or compatible end mediated ligation capture (onto a bead or into a plasmid) is produced. If the restriction site is not naturally present in the genome, the only fragment that should be isolated is the one flanked by the introduced sites. Any naturally occurring restriction sites that are spaced farther apart than the fragments for targeted isolation should not interfere with the process due to use of, for example, size selection. In particular embodiments, less than every 100 kb is reasonable. In particular embodiments, restriction sites need not be used and the whole genome can be sequenced.

Exemplary restriction sites include sites for homing endonucleases, which are a type of endonuclease that cuts DNA upon recognition of a large specific sequence (12-40 bp). Use of a restriction enzyme with a large recognition sequence can help minimize the likelihood that the enzyme will cut DNA at unintended sites. For example, the likelihood is one in one billion that a random sequence will match any given recognition sequence that is 15 bp long. One appropriate restriction site for use is I-SceI. Additional examples of restriction sites include DNA sequences recognized by Sfi I, Acci, Afl III, SapI, Ple I, Tsp45 I, ScrF I, Tse I, PpuM I, Rsr II, and SgrA I.

Genetic constructs encoding markers can be incorporated into parental genomes using any appropriate insertion method. Particular gene editing agents include transcription activator-like effector nucleases (TALENs). TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. The FokI domain functions as a dimer requiring two constructs with unique DNA binding domains for sites on the target sequence. The FokI cleavage domain cleaves within a five or six base pair spacer sequence separating the two inverted half-sites.

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments utilize zinc finger nucleases (ZFNs) as gene editing agents. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, homologous recombination or non-homologous end joining takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, FokI endonuclease.

Guide RNA can be used, for example, with gene-editing agents such as CRISPR-Cas systems. CRISPR-Cas systems include CRISPR repeats and a set of CRISPR-associated genes (Cas). See, for example, Mans et al. (FEMS Yeast Res. 15(2), 2015; doi: 10.1093/femsyr/fov004); DiCarlo et al. (NAR 1-8, 2013; doi:10.1093/nar/gkt135); Laughery et al. (Yeast, 32(12):711-720, 2015; doi: 10.1002/yea.3098);

The CRISPR repeats (clustered regularly interspaced short palindromic repeats) include a cluster of short direct repeats separated by spacers of short variable sequences of similar size as the repeats. The repeats range in size from 24 to 48 base pairs and have some dyad symmetry which implies the formation of a secondary structure, such as a hairpin, although the repeats are not truly palindromic. The spacers, separating the repeats, match exactly the sequences from prokaryotic viruses, plasmids, and transposons. The Cas genes encode nucleases, helicases, RNA-binding proteins, and a polymerase that unwind and cut DNA. Cas1, Cas2, and Cas9 are examples of Cas genes.

At least three different Cas9 nucleases have been developed for genome editing. The first is the wild type Cas9 which introduces DSBs at a specific DNA site, resulting in the activation of DSB repair machinery. DSBs can be repaired by the NHEJ pathway or by homology-directed repair (HDR) pathway. The second is a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

As indicated, the parental marker aspect of the disclosure can be used to identify and select offspring that have genetic recombination events in a specific area of the genome. In particular embodiments, identification of individuals harboring such genetic recombination events can be used for the purpose of improving the efficiency of genetic mapping. Genetic “fine mapping” experiments seek to identify the causative gene(s) that contribute to a trait with a genomic region that contains many genes. In these studies only the small proportion of the progeny resulting from a cross (for instance, those that contain a recombination event within the area of interest) are informative for refining this interval. Thus, selecting individuals that harbor a recombination event in the area (at the outset) improves the efficiency of these experiments by reducing the number of individual progeny that need to be produced, genotyped, phenotyped, and maintained.

In this particular example, and for illustrative purposes, there can be an organism (e.g. yeast) with a phenotypic trait of interest (e.g. heat tolerance). The gene leading to the phenotypic trait of interest is believed to be within a particular area of the genome (“genomic region of interest”).

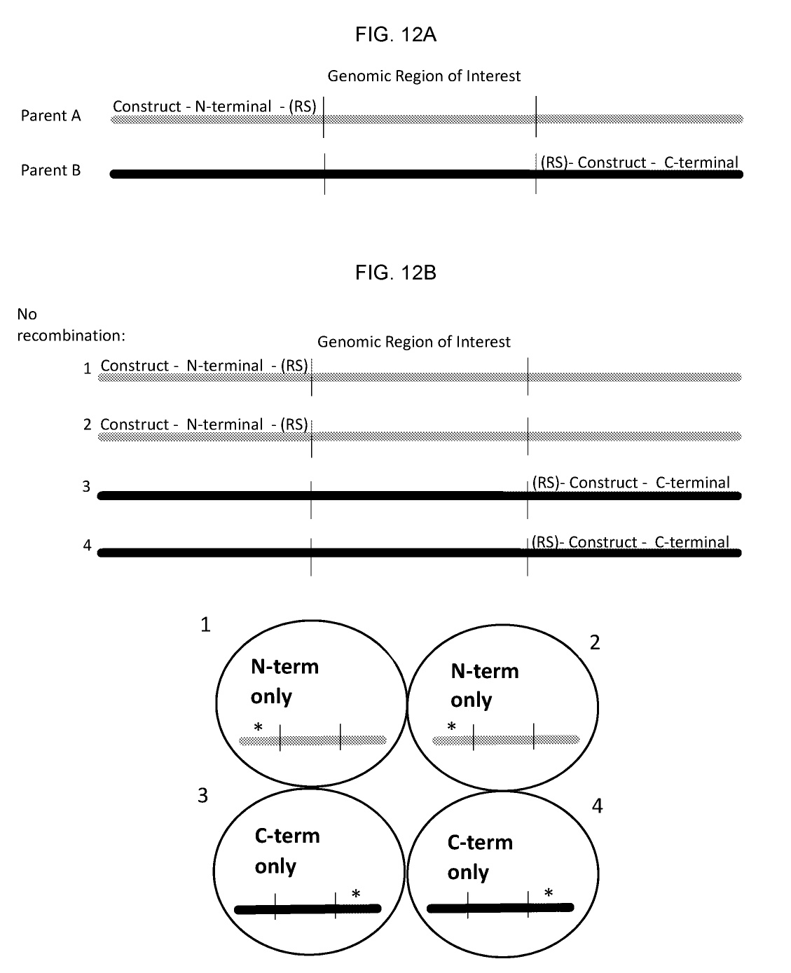

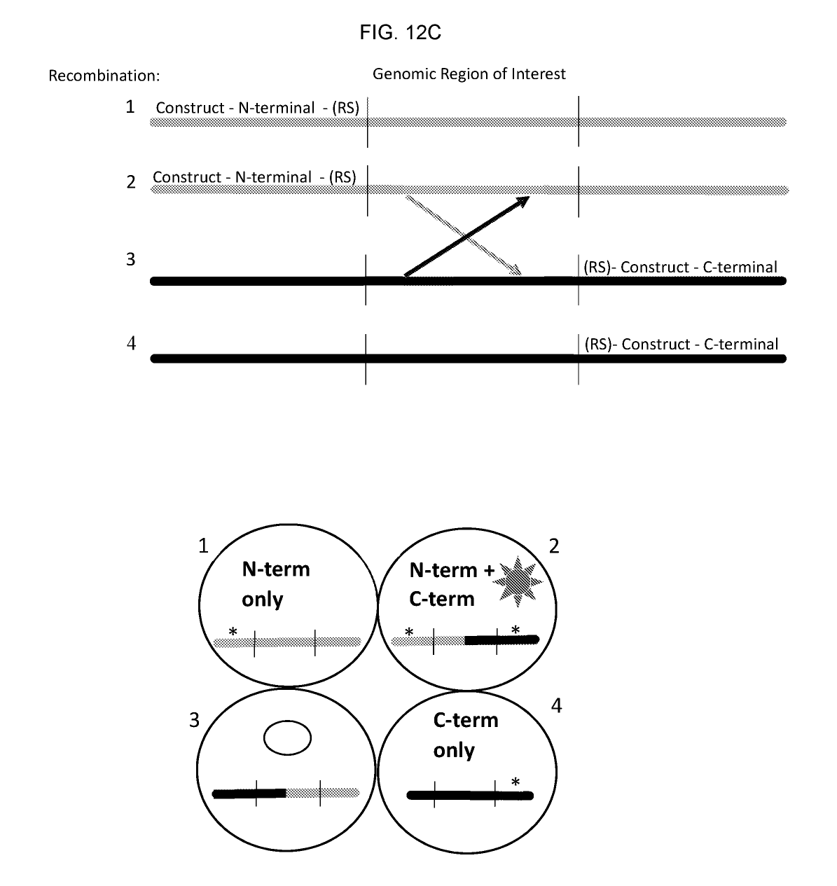

In FIG. 12A, a portion of the haploid genome of Parent 1 with a phenotypic trait of interest is shown as “1”. Within a defined number of base pairs 5′ (or 3′) of the genomic region of interest, a marker construct is inserted. In the depicted embodiment, the genetic construct includes or encodes (i) a promoter, (ii) an interaction domain, (iii) an N-terminal fragment of a split marker (or the C-terminal fragment), (iv) a restriction site (RS), and (v) a termination signal.

Again referring to FIG. 12A, a portion of the haploid genome of Parent 2 without a phenotypic trait of interest is shown as “3”. Within a defined number of base pairs 3′ (or 5′) of the genomic region of interest, a construct is inserted. In the depicted embodiment, the genetic construct includes or encodes (i) a restriction site (RS), (ii) a promoter, (iii) an interaction domain, (iv) the respective complementary N- or C-terminal fragment of the split marker, and (v) a termination signal.

As depicted in FIG. 12B, when the two parents are crossed (that is, bred), the two chromosomes duplicate at the beginning of meiosis. If recombination does not occur within the genomic region of interest, the haploid progeny (products of meiosis) will harbor and express either the N-terminal construct or the C-terminal construct, but not both. Because differential signal creation requires the expression of both the N-terminal and C-terminal portions of the split marker within the same cell, no differential signal will be observed in any of the four meiotic progeny.

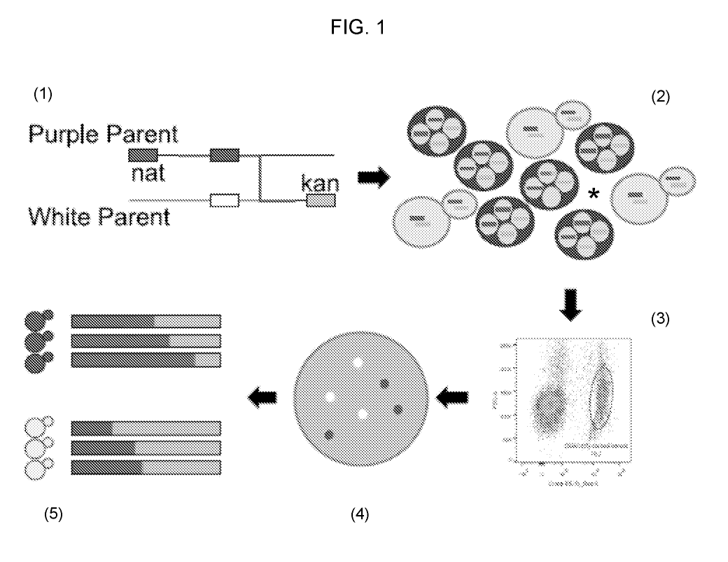

As shown in FIG. 12C, however, if a genetic recombination occurs within the genomic region of interest, both the N-terminal fragment and C-terminal fragment constructs will appear in one of the four haploid progeny cells and that cell will produce a differential signal. It is noted that any odd number of recombination events that occur between the encoding sequences for the N-terminal fragment and C-terminal fragment constructs will likewise result in producing a spore with a differential signal. In the rare event of two recombination events both occurring in the genomic region of interest, it is expected that no differential signal would result. See also the starred tetrads in FIGS. 1(2) and 18 which depict a genetic recombination event.

In particular embodiments utilizing complete, rather than split markers, each genetic construct encodes a complete marker (e.g., one of the drug markers Kan or Nat). Recombinant progeny that have had a recombination event in the genomic region of interest have a differential signal in that they include both drug markers.

In particular embodiments, different genetic constructs can encode fluorescent proteins of different colors (e.g., full length GFP and YFP). Here, recombination events in the genomic region of interest would be indicated by the presence of both green and yellow signals.

The sequences of sorted offspring having recombination events within the genomic region of interest with different phenotypic traits can then be compared, providing faster and cheaper identification of genes of interest.

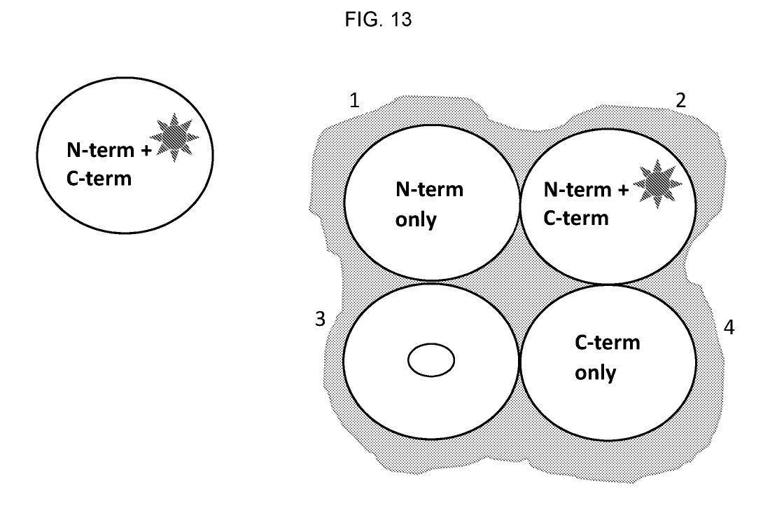

An additional useful property of tetrads and the disclosed systems and methods is that every time there is a recombination event (e.g. the one that produces, for example, a nat-kan double) in a region that gets packaged into one of the spores, the reciprocal recombination product is packaged into one of its sister spores. In the cases where there is a differential signal in the original strains (e.g. a single drug marker), it enables an additional feature—the ability to isolate recombination events in a strain that has no genetic modification (spore 3 in FIGS. 12 and 13). This is valuable because the new strain is non-GMO (important to food or products that will be released to the environment, e.g., bioremediation). This feature is also convenient in laboratory research where there can be a limited pallet of markers (e.g., drugs and fluorescence). More particularly, this feature allows re-use to further refine the strains.

This method greatly enhances efficiency of identifying the gene(s) within an area that confer phenotypic traits of interest. As indicated, and optionally, a restriction site at the opposite end of the genomic region of interest (* in FIG. 12C) can be included to provide restriction sites to cut the genome in spore 3. Note that the unique (or extremely rare) restriction sites flanking the candidate genes in Spore (2) allow cutting of the genome to the area of interest so that shorter segments require sequencing, saving additional resources over whole genome sequencing. Placing restriction sites such that they are proximal to the genomic region of interest (in the diagram above, 3′ of the N-terminal construct and 5′ of the C-terminal construct) allows the genomic region of interest to be excised from recombinant progeny without having to sequence the construct DNA. Given the efficiency with which whole genome sequencing can be performed, however, this feature is optional for organisms with relatively small genomes (e.g. S. cerevisiae) but may provide substantial cost savings for larger genomes (e.g. plant genomes).

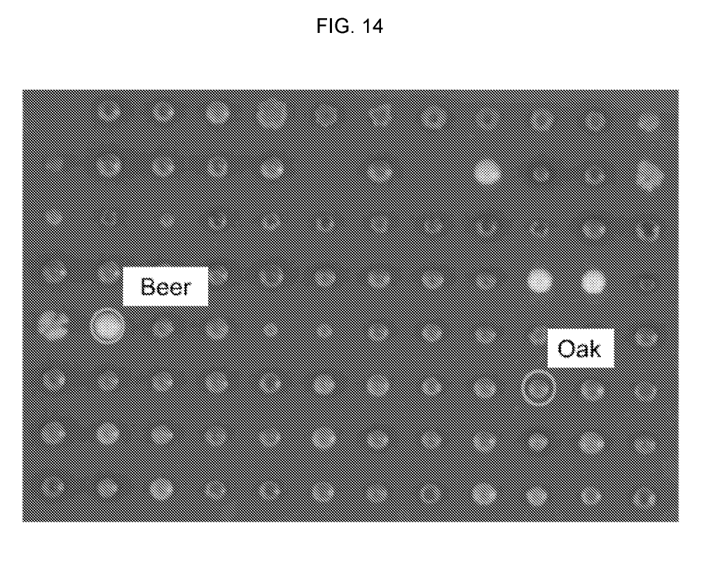

As shown in FIG. 14, most natural isolates of Saccharomyces cerevisiae turn a distinct purple (dark gray in FIGs) on CHROMagar Candida, but some strains remain white (light gray in FIGs). To demonstrate the utility of the disclosed fine mapping method, a yeast cross between a purple parent and a white parent were constructed and the gene(s) linked to this dimorphic trait were mapped. A diploid strain was constructed mating haploid strains derived from IL-01 (phenotypically represented in FIG. 14 by the circled purple colony labeled “Oak”) and CLIB382r (phenotypically represented in FIG. 14 by the circled white colony labeled “Beer”) (Schacherer et al., 2009 Nature 458: 342-345; Cromie et al., 2013 Genomic Sequence Diversity and Population Structure of Saccharomyces cerevisiae Assessed by RAD-seq. G3 (Bethesda)).

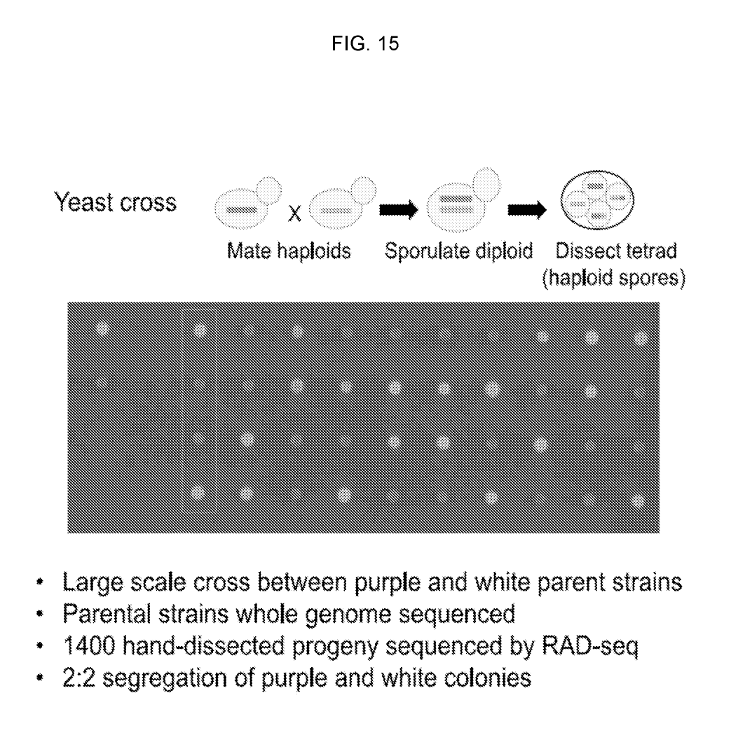

Segregation pattern of the purple and white phenotype among the progeny of a yeast cross is indicative of a monogenic trait. The top section of FIG. 15 depicts the steps of a yeast cross; a population of heterozygous diploids derived by mating the two parents of interest (e.g. IL-01 and CLIB382r) is sporulated, resulting in individual tetrads which each contain the four recombinant progeny of a single meiotic event. The image included in FIG. 15 shows individual S. cerevisiae colonies grown on CHROMagar Candida including the parents of the cross and a sampling of the 1336 progeny obtained from hand-dissecting tetrads. The white parent (CLIB382r) and purple parent (IL-01) of the cross are shown in the upper corner of the image while the four sister-spores from individual tetrads are arrayed in columns across the plate. The Mendelian (2:2) segregation of the purple and white phenotype among the progeny indicates, in this genetic background, a single gene is linked to the colorimetric trait.

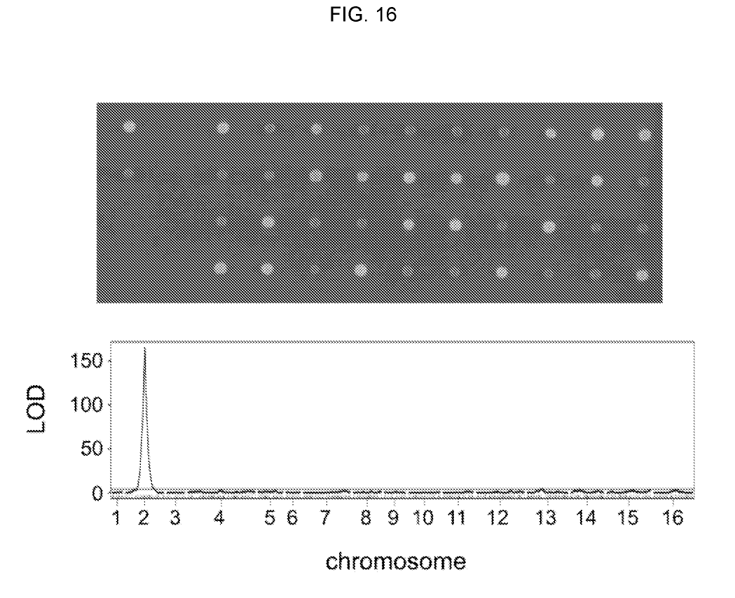

The development of purple color on CHROMagar Candida maps to a region on chromosome II. Prior to applying the disclosed fine mapping method, the broad genomic region(s) linked to the trait must be identified. In this example, a widely-used quantitative trait locus (QTL) mapping approach based on linkage analysis was selected (Lander & Botstein, 1989 Genetics 121: 185-199); however, any method that identifies genetic regions associated with a trait of interest could be used. The colorimetric phenotype was assayed using custom automated image analysis software that extracted color values from images of all progeny after 2 days growth on CHROMagar Candida at 30° C.; although, scoring phenotypes by eye would be sufficient. Both parental strains (IL-01 and CLIB382r) were whole genome sequenced (Cromie et al., 2013 Genomic Sequence Diversity and Population Structure of Saccharomyces cerevisiae Assessed by RAD-seq. G3 (Bethesda)), and all progeny were sequenced using RAD-seq (Baird et al., PLoS One 3: e3376, 2008; Sirr et al., 2015 Genetics 199: 247-262). RAD-seq is a cost-effective method that sequences the same 1% of the genome in all strains defining a set of genomic markers for QTL mapping. As demonstrated by the plot shown in FIG. 16, QTL mapping identified a single major-effect QTL peak on chromosome II linked to the purple phenotype. The LOD (logarithm of odds) score of 159 far exceeds the significance threshold of LOD 4, indicating a very high degree of likelihood that the region under the peak contains the causative gene.

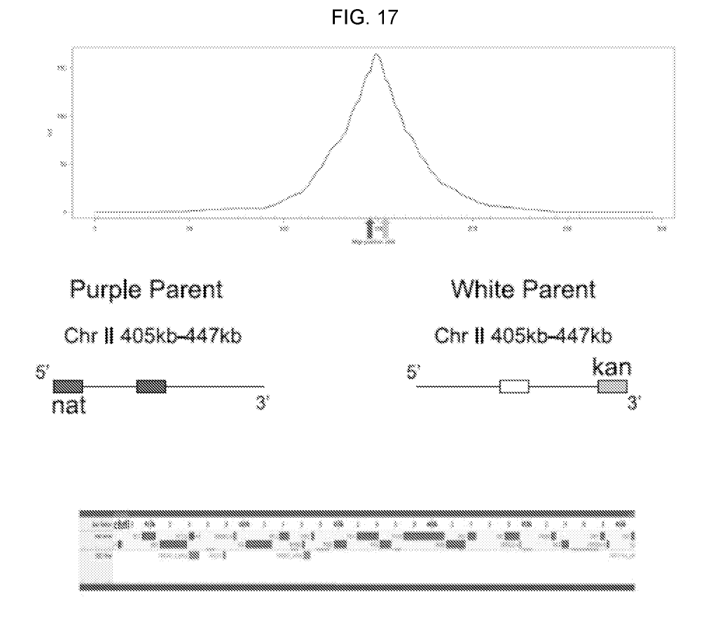

FIG. 17 provides a close-up view of the QTL peak identified on chromosome II from FIG. 16. The x-axis indicates the genomic region on chromosome II with distance in centiMorgans (cM), and the y-axis indicates the LOD score of the peak. While the LOD score of 159 is highly significant, the 1.5 LOD support interval includes a 42 kb region with 30 genes extending from the marker positions 405685 to 447286 (nucleotide positions on chromosome II). A representation of genes (http://chromozoom.org/) included within this interval is shown in FIG. 17. In order to narrow this region to the causative gene or polymorphism using the disclosed fine mapping method, the region was first flanked by integrating a selectable drug marker (natMX4) (Goldstein & Mccusker, 1999 Yeast 15: 1541-1553) at the 5′ end of the interval in the purple parent (YO2302) and integrating a second selectable drug marker (kanMX4) (Wach et al., 1994 Yeast 10: 1793-1808) at the 3′ end of the interval in the white parent (YO2308). This cross is henceforth identified as the N-K cross. To avoid generating a biased sampling of crossover events, a second diploid using a set of reciprocally marked strains was also constructed. For the reciprocal cross the 3′ end of the interval was marked with kanMX4 in the purple parent (YO2304) and the 5′ end of the interval was marked with natMX4 in the white parent (YO2306). This cross is henceforth identified as the K-N cross. Strains were constructed using standard methods, and diploids were selected on YPD supplemented with standard concentrations of G418 and nourseothricin (Rose et al., 1990 Methods in yeast genetics: a laboratory course manual. Cold Spring Harbor Laboratory Press, Cold Spring Harbor, N.Y.).

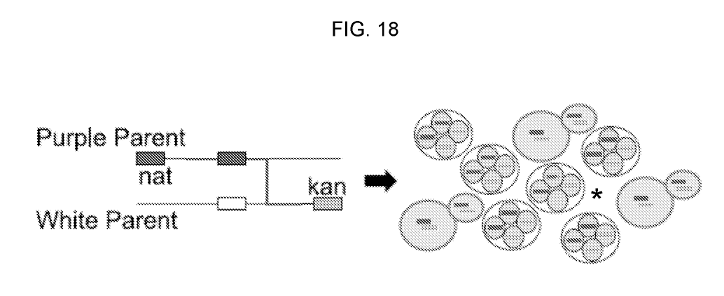

FIG. 18 depicts the DNA region marked by the natMX4 (dark gray rectangle) and kanMX4 (light gray rectangle) drug cassettes delineating the fine mapping region. An informative recombination event requires a crossover (dark line connecting the two parental chromosomes) within the marked region, thereby linking the causal polymorphism (black or white rectangles) to both drug markers. During meiosis I (MI), a crossover within the fine mapping region generally results in a tetrad as depicted by the starred tetrad within FIG. 18: one spore inherits both natMX4 and kanMX4 markers (represented by the mixed dark gray and light gray rectangle within the spore), one spore inherits neither drug marker (represented by the empty spore), and two spores, with no crossover in the region, inherit the original parental haplotypes and are thus marked with only natMX4 or kanMX4 cassette (represented by the dark gray or light gray rectangles). The informative progeny carried forward in this example inherit both drug markers; however, it is noteworthy that unmarked strains also harbor informative crossovers and can be selected based on sensitivity to both G418 and nourseothricin.

As recombination can occur at many different locations throughout the genome, an event within the fine mapping region may occur at low frequency among a population of diploids undergoing meiosis. In order to provide the statistical mapping power required to narrow the QTL to a single gene, this fine mapping method overcomes this constraint by selecting and isolating spores with an informative recombination from non-informative spores and unsporulated diploids. To this end, individual diploid colonies of the N-K cross and the K-N cross were grown overnight in 3 mL YPD cultures at 30° C., and the cell pellets were sporulated (Ludlow et al., 2013 Nat Methods. 10: 671-675; Scott et al., 2014 J Vis Exp. 87: 51401). Tetrads were stained using DiBAC4(5) (Anaspec AS-84701) as follows: 1 mL sporulation culture was washed once in phosphate buffered saline (PBS) and resuspended and stained for 1 minute in the dark in 1 mL PBS with a final concentration of 1 μg/mL DiBAC4(5); cells were washed twice in 1 mL PBS, resuspended in 5 ml PBS and briefly sonicated. Using a Sony LE-SH800 sorter with the 561 nm laser and FL3 filter set (617/30), tetrads were separated from dyads by gating the far lower right population on FSC-W/FSC-H and the high FSC-W/PE population (FIG. 3). For each cross (N-K and K-N), tetrads were sorted onto 8 YPD plates (200 tetrads per plate) supplemented with G418 and nourseothricin (3.2×10³ tetrads in total). Spores were disrupted and plated as described previously (Ludlow et al., 2013 Nat Methods. 10: 671-675; Scott et al., 2014 J Vis Exp. 87: 51401) and grown overnight at 30° C. Individual colonies (96 strains from each cross) were then picked and grown in YPD in 96-well plates for 2 days at 30° C. Progeny (all of which inherited both the kanMX4 and natMX4 cassettes) were pinned to CHROMagar Candida Omni-tray (Thermo Scientific) and grown 24 hours at 30° C. for phenotyping. The colorimetric phenotype was assayed using custom automated image analysis software that extracted color values from images of all progeny after 2 days growth on CHROMagar Candida at 30° C.; however, scoring phenotypes by eye would be sufficient. Strains grown in this same 96-well format were grouped into 4 pools for genotyping. All strains included in pools contained both drug marker cassettes, and equal numbers of purple and white strains were selected for pools. Pool 1 included 74 purple strains from cross N-K. Pool 2 included 14 purple strains from cross K-N. Pool 3 included 14 white strains from cross N-K, and Pool 4 included 74 white strains from cross K-N. While only the 42 kb fine mapping region required sequencing, it proved cost-effective to whole genome sequence each of the 4 pools using standard Illumina methods (https://www.illumina.com/).

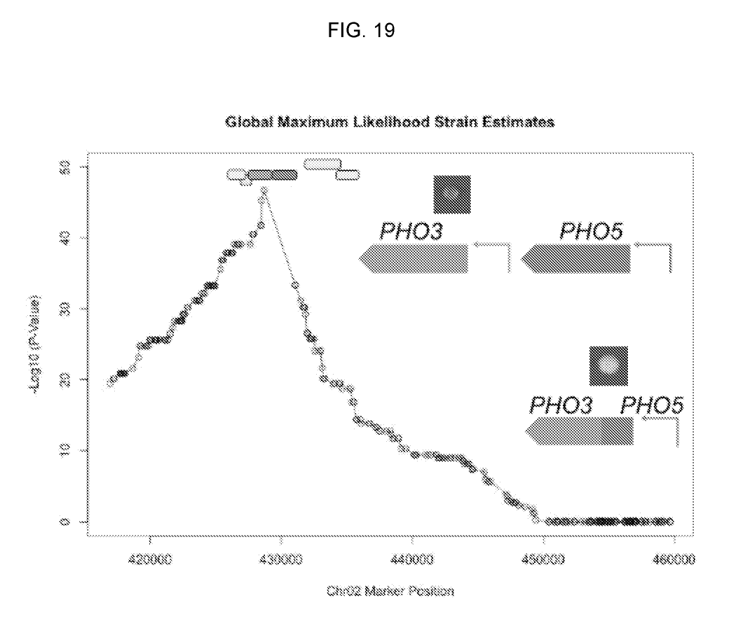

The only region of the genome expected to deviate significantly from a 50:50 segregation pattern, is the region linked to the colorimetric trait. Thus, the global maximum likelihood strain estimate plot depicted in FIG. 19 indicates the genetic region most likely associated with the purple phenotype. Circles on the plot depict individual RAD-seq markers and their relative likelihood (p-value) of being linked to the purple phenotype. Ovals at the top depict the genes within the region of the markers. Fine mapping results identify the causative genes of the purple trait on CHROMagar Candida as the tandemly arrayed PHO3 and PHO5 acid phosphatases (colored gray ovals). PHO3 and PHO5 share 87% amino acid sequence homology (Bajwa et al., 1984 Nucleic Acids Res 12: 7721-7739); however, they are differentially regulated. PHO3 is expressed regardless of internal phosphate concentration while PHO5, known as the repressible acid phosphatase, is expressed only in phosphate limiting conditions (Nosaka et al., 1989 FEMS Microbiol Lett 51: 55-59; O'Neill et al., 1996 Science 271: 209-212; Sambuk et al., 2011 Acid phosphatases of budding yeast as a model of choice for transcription regulation research. Enzyme Res 2011: 356093). As this region is highly homologous, whole genome sequencing of the parent strains was ambiguous. Interestingly, Sanger sequencing (https://www.genewiz.com/) of the white parent's PHO3/PHO5 revealed a loopout in which most of the PHO5 coding region and the entire PHO3 promoter region was deleted. The resulting gene fusion between PHO3 and PHO5 is depicted in FIG. 19 under the image of the white parent. The sequence of this chimeric version is very similar to PHO3 but alters the conditions under which the gene is expressed. Notably, deleting this region in the IL-01 background (purple parent) results in an altered white phenotype when grown on CHROMagar Candida, a result that confirms that the fine mapping method correctly identified the gene associated with this colorimetric trait.

Utilizing aspects of Part I of the disclosure, and to further enhance this method in yeast and when the marker or split marker is a fluorescent protein, fluorescent dyes can be used to further isolate tetrads from diploids. For example, because fluorescence constructs are present in unsporulated diploids, marked recombinant progeny and unsporulated diploids fluoresce, thereby confounding the isolation of recombinant tetrads. However, fluorescent dyes are able to accumulate in the interspore area of a tetrad. Using two channel flow cytometry, tetrads can be isolated from diploids by FACS gating based on size and fluorescent dye staining (e.g. red fluorescence). Tetrads within the population that harbor spores with a recombination event in the interval (genomic region of interest) will also be positive for fluorescence conferred by expression of both parts of the fluorescent protein(s) (e.g. green for GFP; FIG. 13). This enhancement of the method can further expedite gene mapping by pre-screening unsporulated diploids out of a mapping analysis.

Fluorescent markers, including fluorescent dyes, have a wide range of absorption/emission profiles. Sorters typically have several filter options to use with each laser so that the user can select narrow bands of the emission profile which helps to separate fluorescent markers that have emission profiles that bleed over into the other markers. DiBAC₄(5) is a red fluorescent dye and a structural analog of the commonly used oxonol, DiBAC₄(3). However, it's emission spectrum has little overlap in the green channel, reducing compensation adjustments required for flow cytometry gating when used in conjunction with green fluorescent markers such as GFP or stains such as FITC (Hernlem and Hua, Curr Microbiol. 61: 57-63, 2010).

Thus, particular embodiments can utilize combinations of fluorescent signals (fluorescent dyes and a unified fluorescent protein) wherein the fluorescent signals are chosen in combinations to reduce or avoid overlap between emission profiles. In particular embodiments, the selected fluorescent signals will have emission wavelength peaks that are separated by at least 50 nm; at least 100 nm; at least 150 nm; or at least 200 nm. For example, the emission wavelength peak of DiBAC4(5) is 616 nm and GFP's emission wavelength peak is 510. Propidium iodide (PI) has an emission wavelength peak similar to DiBAC4(5), and in particular embodiments is beneficially used in combination with GFP. In particular embodiments, DiBAC4(3) can be used in combination with Red Fluorescent Protein (RFP). The emission wavelength peak of DiBAC4(3) is 516 nm and RFP's emission wavelength peak is 584.

Thus, at least three aspects of the described method can create significant efficiencies alone or in combination: (1) identification of offspring with a genetic recombination within the genomic region of interest; (2) restriction sites inserted around the genomic region of interest to shorten the length of genome requiring sequencing; and (3) isolation of recombinant tetrads from unsporulated diploids.

Exemplary data-processing architecture. Aspects of the current disclosure are described in terms of algorithms and/or symbolic representations of operations on data bits and/or binary digital signals stored within a computing system, such as within a computer and/or computing system memory. These algorithmic descriptions and/or representations are the techniques used by those of ordinary skill in the data processing arts to convey the substance of their work to others skilled in the art. An algorithm is here, and generally, considered to be, a self-consistent sequence of operations and/or similar processing leading to a desired result. The operations and/or processing may involve physical manipulations of physical quantities. Typically, although not necessarily, these quantities may take the form of electrical and/or magnetic signals capable of being stored, transferred, combined, compared and/or otherwise manipulated. It has proven convenient, at times, principally for reasons of common usage, to refer to these signals as bits, messages, data, values, elements, symbols, characters, terms, numbers, numerals and/or the like. It should be understood, however, that all of these and similar terms are to be associated with appropriate physical quantities and are merely convenient labels.

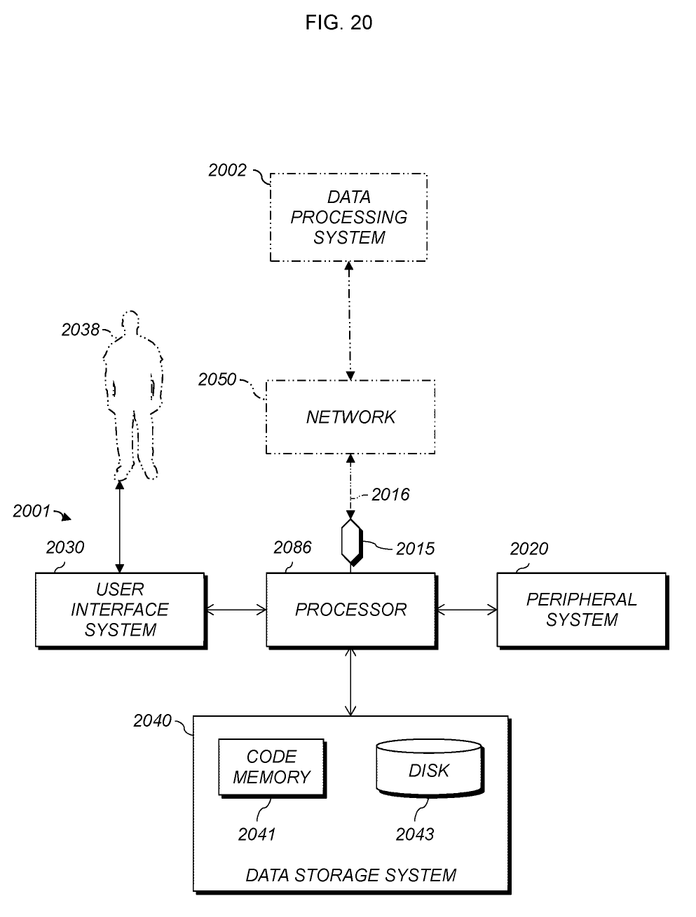

Particular embodiments disclosed herein may be practiced utilizing computing systems. Computing systems can be configured to receive, store, and analyze data (e.g., genetic sequence data, image data, fluorescent data). Computing systems may receive data via a network. FIG. 20 depicts is a high-level diagram showing components of a data-processing system 2001 for analyzing data and performing other analyses described herein, and related components. The system 2001 may include a processor 2086, a peripheral system 2020, a user interface system 2030, and a data storage system 2040. The peripheral system 2020, the user interface system 2030 and the data storage system 2040 are communicatively connected to the processor 2086. Processor 2086 can be communicatively connected to network 2050 (shown in phantom), e.g., the Internet or other communications network, as discussed below. As used herein, the term “device” can refer to any one or more of processor 2086, peripheral system 2020, user interface system 2030, data storage system 2040. Any of these, or other devices, can each connect to one or more network(s) 2050. Processor 2086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 2086 can implement processes of various aspects described herein. Processor 2086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise.

The phrase “communicatively connected” includes any type of connection, wired or wireless, for communicating data between devices or processors. These devices or processors can be located in physical proximity or not. For example, subsystems such as peripheral system 2020, user interface system 2030, and data storage system 2040 are shown separately from the data processing system 2086 but can be stored completely or partially within the data processing system 2086.

The peripheral system 2020 can include or be communicatively connected with one or more devices configured or otherwise adapted to provide digital content records to the processor 2086 or to take action in response to processor 186. For example, the peripheral system 2020 can include digital still cameras, digital video cameras, DNA sequencers, flow cytometers, or other data generating equipment. The processor 2086, upon receipt of digital content from a device in the peripheral system 2020, can store such digital content in the data storage system 2040.

The user interface system 2030 can convey information in either direction, or in both directions, between a user 2038 and the processor 2086 or other components of system 2001. The user interface system 2030 can include a mouse, a keyboard, another computer (connected, e.g., via a network or a null-modem cable), or any device or combination of devices from which data is input to the processor 2086. The user interface system 2030 also can include a display device, a printer, a processor-accessible memory, or any device or combination of devices to which data is output by the processor 2086. The user interface system 2030 and the data storage system 2040 can share a processor-accessible memory.

In various aspects, processor 2086 includes or is connected to communication interface 2015 that is coupled via network link 2016 (shown in phantom) to network 2050. For example, communication interface 2015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 2015 sends and receives electrical, electromagnetic or optical signals that carry digital or analog data streams representing various types of information across network link 2016 to network 2050. Network link 2016 can be connected to network 2050 via a switch, gateway, hub, router, or other networking device.

In various aspects, system 2001 can communicate, e.g., via network 2050, with a data processing system 2002, which can include the same types of components as system 2001 but is not required to be identical thereto. Systems 2001, 2002 are communicatively connected via the network 2050. Each system 2001, 2002 executes computer program instructions to carry out functions disclosed herein.

Processor 2086 can send messages and receive data, including program code, through network 2050, network link 2016 and communication interface 2015. For example, a server can store requested code for an application program (e.g., a JAVA applet) on a tangible non-volatile computer-readable storage medium to which it is connected. The server can retrieve the code from the medium and transmit it through network 2050 to communication interface 2015. The received code can be executed by processor 2086 as it is received, or stored in data storage system 2040 for later execution.

Data storage system 2040 can include or be communicatively connected with one or more processor-accessible memories configured or otherwise adapted to store information. The memories can be internal, e.g., within a chassis, or as parts of a distributed system. The phrase “processor-accessible memory” is intended to include any data storage device to or from which processor 2086 can transfer data (using appropriate components of peripheral system 2020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), erasable programmable read-only memories (EPROM, EEPROM, or Flash), and random-access memories (RAMs). One of the processor-accessible memories in the data storage system 2040 can be a tangible non-transitory computer-readable storage medium, i.e. a non-transitory device or article of manufacture that participates in storing instructions that can be provided to processor 2086 for execution.

In an example, data storage system 2040 includes code memory 2041, e.g., a RAM, and disk 2043, e.g., a tangible computer-readable storage device or medium such as a hard drive. Computer program instructions are read into code memory 2041 from disk 2043. Processor 2086 then executes one or more sequences of the computer program instructions loaded into code memory 2041, as a result performing process steps described herein. In this way, processor 2086 carries out a computer implemented process. For example, steps of methods 900, 1000, 1100, 1102, 1104, and 1106 described herein, blocks of the flowchart illustrations or block diagrams herein, and combinations of those, can be implemented by computer program instructions. Code memory 2041 can also store data or can store only code. In some examples, at least one of code memory 2041 or disk 2043 can be or include a computer-readable medium (CRM), e.g., a tangible non-transitory computer storage medium.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects These aspects can all generally be referred to herein as a “service,” “circuit,” “circuitry,” “module,” or “system.”

Furthermore, various aspects herein may be embodied as computer program products including computer readable program code (“program code”) stored on a computer readable medium, e.g., a tangible non-transitory computer storage medium or a communication medium. A computer storage medium can include tangible storage units such as volatile memory, nonvolatile memory, or other persistent or auxiliary computer storage media, removable and non-removable computer storage media implemented in any method or technology for storage of information such as computer-readable instructions, data structures, program modules, or other data. A computer storage medium can be manufactured as is conventional for such articles, e.g., by pressing a CD-ROM or electronically writing data into a Flash memory. In contrast to computer storage media, communication media may embody computer-readable instructions, data structures, program modules, or other data in a modulated data signal, such as a carrier wave or other transmission mechanism. As defined herein, computer storage media do not include communication media. That is, computer storage media do not include communications media consisting solely of a modulated data signal, a carrier wave, or a propagated signal, per se.

The program code includes computer program instructions that can be loaded into processor 2086 (and possibly also other processors), and that, when loaded into processor 2086, cause functions, acts, or operational steps of various aspects herein to be performed by processor 2086 (or other processor). Computer program code for carrying out operations for various aspects described herein may be written in any combination of one or more programming language(s), and can be loaded from disk 2043 into code memory 2041 for execution. The program code may execute, e.g., entirely on processor 2086, partly on processor 2086 and partly on a remote computer connected to network 2050, or entirely on the remote computer.

In some examples, processor(s) 2086 and, if required, data storage system 2040 or portions thereof, are referred to for brevity herein as a “control unit.” For example, a control unit can include a CPU or DSP and a computer storage medium or other tangible, non-transitory computer-readable medium storing instructions executable by that CPU or DSP to cause that CPU or DSP to perform functions described herein. Additionally or alternatively, a control unit can include an ASIC, FPGA, or other logic device(s) wired (e.g., physically, or via blown fuses or logic-cell configuration data) to perform functions described herein.

In some examples, a “control unit” as described herein includes processor(s) 2086. A control unit can also include, if required, data storage system 2040 or portions thereof. For example, a control unit can include a CPU or DSP and a computer storage medium or other tangible, non-transitory computer-readable medium storing instructions executable by that CPU or DSP to cause that CPU or DSP to perform functions described herein. Additionally or alternatively, a control unit can include an ASIC, FPGA, or other logic device(s) wired (e.g., physically, or via blown fuses or logic-cell configuration data) to perform functions described herein. In some examples of control units including ASICs or other devices physically configured to perform operations described herein, a control unit does not include computer-readable media storing executable instructions.

Computing systems can belong to, or include, a variety of categories or classes of devices such as traditional server-type devices, desktop computer-type devices, mobile-type devices, special purpose-type devices, and/or embedded-type devices.

### Exemplary Embodiments

1. Use of a fluorescent dye to sort tetrads from vegetative cells, dyads, and dead cells.
2. Use of a fluorescent dye in a synthetic lethality screen.
3. A use of embodiment 1 or 2 in combination with fluorescence-activated cell sorting (FACS).
4. A use of any of embodiments 1-3 wherein the fluorescent dye is selected from xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′,5′ dichloro 2′,7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzamide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, Tetramethylrhodamine, Lissamine, or Napthofluorescein.
5. A use of any of embodiments 1-3 wherein the fluorescent dye is a vital dye.
6. A use of embodiment 5 wherein the vital dye is selected from Bis-(1,3-dibutylbarbituric acid) pentamethine oxonol; Anaspec AS-84701, calcein AM, carboxyfluorescein diacetate, copper phthalocyanine tetrasulfonate, DiOC (3,3′-dihexyloxacarbocyanine iodide), Evans blue, gadolinium texaphyrin, indocyanine green monosodium salt, isosulfan, methylene blue, Nile red, patent blue V, patent blue VF, propodium iodide, rhodamine 123, and sulfobromophthaleine.
7. A use of embodiment 5 wherein the vital dye is pentamethine oxonol or propodium iodide.
8. A use of embodiment 3 wherein the FACS utilizes fluorescence intensity to sort tetrads, dyads, and dead cells away from live vegetative cells.
9. A use of embodiment 3 or 8 wherein the FACS utilizes 488 nm emission and a 595LP 610/20 filter.
10. A use of embodiment 3, 8 or 9 wherein the FACS gates the tetrad, dyad, and dead cell population using forward scatter.
11. A method of sorting tetrads from vegetative cells, dyads, and dead cells, the method including:

incubating a mixture of tetrads, vegetative cells, dyads, and dead cells in a fluorescent dye solution to produce a stained mixture of cells; and

sorting the mixture of stained cells based on an optical characteristic attributable to the fluorescent dye,

thereby sorting the tetrads from the vegetative cells, dyads, and dead cells.
12. A method of embodiment 11 wherein the fluorescent dye solution includes a xanthene dye, fluorescein dye, rhodamine dye, FITC, FAM, HEX, JOE, TAMRA, ROX, R6G5, R6G6, rhodamine 110; cyanine dye, Cy3, Cy5 Cy7; Alexa dye, Alexa-fluor-555; coumarin, Diethylaminocoumarin, umbelliferone; benzamide dye, Hoechst 33258; phenanthridine dye, Texas Red; ethidium dye; acridine dye; carbazole dye; phenoxazine dye; porphyrin dye; polymethine dye, BODIPY dye, quinoline dye, Pyrene, Fluorescein Chlorotriazinyl, R110, Eosin, Tetramethylrhodamine, Lissamine, or Napthofluorescein.
13. A method of embodiment 12 wherein the fluorescent dye is a vital dye.
14. A method of embodiment 13 wherein the vital dye is selected from Bis-(1,3-dibutylbarbituric acid) pentamethine oxonol; Anaspec AS-84701, calcein AM, carboxyfluorescein diacetate, copper phthalocyanine tetrasulfonate, DiOC (3,3′-dihexyloxacarbocyanine iodide), Evans blue, gadolinium texaphyrin, indocyanine green monosodium salt, isosulfan, methylene blue, Nile red, patent blue V, patent blue VF, propodium iodide, rhodamine 123, and sulfobromophthaleine.
15. A method of embodiment 13 wherein the vital dye is pentamethine oxonol or propodium iodide.
16. A method of any of embodiments 11-15 wherein the sorting is FACS-based sorting.
17. A method of embodiment 16 wherein the FACS-based sorting utilizes fluorescence intensity to sort tetrads, dyads, and dead cells away from live vegetative cells.
18. A method of embodiment 16 or 17 wherein the FACS-based sorting utilizes 488 nm emission and a 595LP 610/20 filter.
19. A method of any of embodiments 16-18 wherein the FACS-based sorting gates the tetrad, dyad, and dead cell population using forward scatter.
20. A method of performing a synthetic lethality screen including:

incubating a mixture of tetrads, vegetative cells, dyads, and dead cells in a fluorescent dye solution to produce a stained mixture of cells; and

identifying tetrads with at least one dead spore based on an optical characteristic attributable to the fluorescent dye,

thereby performing the synthetic lethality screen.
21. A method of embodiment 20 further including sorting tetrads with at least one dead spore from other tetrads, vegetative cells, dyads, and dead cells based on an optical characteristic attributable to the fluorescent dye.
22. A method of embodiment 20 or 21 wherein the fluorescent dye solution includes a xanthene dye, fluorescein dye, rhodamine dye, FITC, FAM, HEX, JOE, TAMRA, ROX, R6G5, R6G6, rhodamine 110; cyanine dye, Cy3, Cy5 Cy7; Alexa dye, Alexa-fluor-555; coumarin, Diethylaminocoumarin, umbelliferone; benzamide dye, Hoechst 33258; phenanthridine dye, Texas Red; ethidium dye; acridine dye; carbazole dye; phenoxazine dye; porphyrin dye; polymethine dye, BODIPY dye, quinoline dye, Pyrene, Fluorescein Chlorotriazinyl, R110, Eosin, Tetramethylrhodamine, Lissamine, or Napthofluorescein.
23. A method of embodiment 20 or 21 wherein the fluorescent dye is a vital dye.
24. A method of embodiment 23 wherein the vital dye is selected from Bis-(1,3-dibutylbarbituric acid) pentamethine oxonol; Anaspec AS-84701, calcein AM, carboxyfluorescein diacetate, copper phthalocyanine tetrasulfonate, DiOC (3,3′-dihexyloxacarbocyanine iodide), Evans blue, gadolinium texaphyrin, indocyanine green monosodium salt, isosulfan, methylene blue, Nile red, patent blue V, patent blue VF, propodium iodide, rhodamine 123, and sulfobromophthaleine.
25. A method of embodiment 23 wherein the vital dye is pentamethine oxonol or propodium iodide.
26. A method of any of embodiments 21-25 wherein the sorting is FACS-based sorting.
27. A method of embodiment 26 wherein the FACS-based sorting utilizes fluorescence intensity to sort tetrads with a dead spore from tetrads having all living spores; dyads; and live vegetative cells.
28. A method of embodiment 26 or 27 wherein the FACS-based sorting utilizes 488 nm emission and a 595LP 610/20 filter.
29. A method of any of embodiments 26-28 wherein the FACS-based sorting gates the cell population using forward scatter.
30. A method of capturing the tetrad relationship of recombinant progeny from a yeast cross using patterns of natural genetic sequences including:

sequencing aspects of the natural genetic sequence of the recombinant progeny; and

grouping recombinant progeny into tetrad relationships based on redundant and mirrored features in the natural genetic sequence of the grouped recombinant progeny.

31. A method of embodiment 30 wherein the aspects of the natural genetic sequence include centromere-linked markers; allele presence; and/or location and/or number of recombination events.
32. A method of embodiment 30 or 31 wherein the sequencing is whole genome sequencing or restriction-associated DNA (RAD) sequencing.
33. A method of embodiment 30 or 31 wherein the sequencing includes sequencing less than 20% of the whole genome; less than 10% of the whole genome; or less than 5% of the whole genome.
34. A method of embodiment 30 or 31 wherein the sequencing includes sequencing 3% of the whole genome.
35. A method of any of embodiments 30-34 wherein grouping recombinant progeny into tetrad relationships requires at least 50% shared valid markers flanking centromeres.
36. A method of any of embodiments 30-34 wherein grouping recombinant progeny into tetrad relationships requires at least 50% shared valid markers flanking centromeres and perfect consensus between these markers.
37. A method of any of embodiments 30-36 further including assessing and/or refining the grouping utilizing mutual information between two or more of the recombinant progeny.
38. A method of any of embodiments 30-37, further including assessing and/or refining the grouping utilizing clustering algorithms, Markov chains, or pattern matching based on reciprocal recombination events.
39. A method of any of embodiments 30-38 further including assessing and/or refining the grouping utilizing delta scores.
40. A method of any of embodiments 30-38 further including assessing and/or refining the grouping by calculating a pair-wise score.
41. A method of any of embodiments 30-40 practiced in combination with a use of embodiments 1-10 and/or a method of embodiments 11-29.
42. A computer readable medium encoding computer-readable instructions that, when executed, cause one or more processors to perform the method of any of embodiments 30-40.
43. A data-processing system including at least one processor and at least one data storage system, the at least one data storage system including computer-readable instructions that, when executed by the at least one processor, cause the data-processing system to perform the method of any of embodiments 30-40.
44. Use of genetic constructs encoding one marker in the first parent of an offspring and a second marker in the second parent of the offspring to identify the occurrence of a genetic recombination event in a genomic region of interest in the offspring.
45. A use of embodiment 44 wherein the first and second marker are of the same type of marker, creating a signal of a different magnitude or intensity when expressed together in the offspring with the genetic recombination event in the genomic region of interest.
46. A use of embodiment 44 wherein the first and second marker are different types of marker, creating a combined signal when expressed together in the offspring with the genetic recombination event in the genomic region of interest.
47. Use of genetic constructs encoding a split marker in the parents of an offspring to identify the occurrence of a genetic recombination event in a genomic region of interest in the offspring.
48. A method of detecting a genetic recombination event or lack thereof in an offspring, the method including:

inserting a first genetic construct encoding a first marker into the genome of a first parent; and

inserting a second genetic construct encoding a second marker into the genome of a second parent; and

evaluating an offspring of the first and second parent for a differential signal created by the combination of the first and second marker in the offspring, wherein detection of the differential signal indicates occurrence of the genetic recombination event.

49. A method of embodiment 48 wherein the genetic recombination event occurs within a genomic region of interest.
50. A method of embodiment 48 or 49 wherein the first marker and/or the second marker is a drug resistance marker, a fluorescent protein, a cerulenin resistance marker or an auxotrophic marker.
51. A method of embodiment 48 or 49 wherein the first marker and/or the second marker is a drug resistance marker or a fluorescent protein.
52. A method of embodiment 48 or 49 wherein the first marker and the second marker are drug resistance markers.
53. A method of embodiment 48 or 49 wherein the first marker and the second marker are fluorescent proteins.
54. A method of any of embodiments 48-53 wherein the first and/or second genetic construct includes a promoter and/or a rare or unique restriction site.
55. A method of embodiment 54 wherein the promoter is pGPD1.
56. A method of embodiment 54 or 55 wherein the restriction site is a homing endonuclease restriction site.
57. A method of detecting a genetic recombination event or lack thereof in an offspring, the method including:

inserting a first genetic construct encoding an aspect of a split marker into the genome of a first parent; and

inserting a second genetic construct encoding a complementary aspect of the split marker into the genome of a second parent; and

evaluating an offspring of the first and second parent for a differential signal created by the aspect and complementary aspect of the split marker, wherein detection of the differential signal indicates occurrence of the genetic recombination event.

58. A method of embodiment 57 wherein the genetic recombination event is recombination within a genomic region of interest.
59. A method of embodiment 57 or 58 wherein the aspect is an N-terminal fragment of a protein and the complementary aspect is the C-terminal fragment of a protein.
60. A method of any of embodiments 57-59 wherein the first and/or second genetic construct includes a promoter, a sequence encoding an interaction domain, and optionally a rare or unique restriction site.
61. A method of embodiment 60 wherein the promoter is pGPD1.
62. A method of embodiment 60 or 61 wherein the interaction domain is EF1 or EF2.
63. A method of any of embodiments 60-62 wherein the restriction site is a homing endonuclease restriction site.
64. A method of any of embodiments 57-63 wherein the differential signal is drug resistance or fluorescence.
65. A method of any of embodiments 48-64 further including incubating offspring in a fluorescent dye solution.
66. A method of embodiment 65 further including separating unsporulated diploids from tetrads having a recombination event.
67. A method of embodiments 65 or 66 wherein the differential signal is fluorescence and the fluorescent emission wavelength peaks of the differential signal and the fluorescent dye are separated by at least 50 nm.
68. A method of embodiments 65 or 66 wherein the differential signal is emitted by GFP or RFP and the fluorescent dye signal is emitted by DiBAC4(5), DiBAC4(3), or Propidium iodide (PI).
69. A method of any of embodiments 48-64 practiced in combination with a use of embodiments 1-10 or 44-47 and/or a method of embodiments 11-40.
70. Chromosomes from a sporulating organism that utilizes meiosis in sexual reproduction, wherein each chromosome is modified with a genetic construct encoding a marker.
71. Chromosomes of embodiment 70 wherein the marker is a drug resistance marker, a fluorescent protein, a cerulenin resistance marker or an auxotrophic marker.
72. Chromosomes of embodiment 70 or 71 wherein different chromosomes include different genetic constructs encoding different markers.
73. Chromosomes of embodiment 72 wherein the different markers are different drug resistance markers.
74. Chromosomes of embodiment 72 wherein the different markers are different fluorescent proteins.
75. Chromosomes of embodiment 72 wherein the different markers are different drug resistance markers and different fluorescent proteins.
76. Chromosomes of any of embodiments 70-75 wherein the genetic constructs include a promoter and/or a rare or unique restriction site.
77. Chromosomes of embodiment 76 wherein the promoter is pGPD1.
78. Chromosomes of embodiment 76 or 77 wherein the restriction site is a homing endonuclease restriction site.
79. Chromosomes from a sporulating organism that utilizes meiosis in sexual reproduction, wherein each chromosome is modified with a genetic construct encoding an aspect of a split marker.
80. Chromosomes of embodiment 79 wherein the aspect encoded by the genetic construct of one chromosome is an N-terminal fragment of a protein and the aspect encoded by the genetic construct of a second chromosome is a complementary C-terminal fragment of the protein.
81. Chromosomes of embodiment 79 or 80 wherein the genetic constructs include a promoter, a sequence encoding an interaction domain, and optionally a rare or unique restriction site.
82. Chromosomes of embodiment 81 wherein the promoter is pGPD1.
83. Chromosomes of embodiment 81 or 82 wherein the interaction domain is EF1 or EF2.
84. Chromosomes of any of embodiments 81-83 wherein the restriction site is a homing endonuclease restriction site.
85. A chromosome from a sporulating organism that utilizes meiosis in sexual reproduction, which chromosome is modified with a genetic construct including:
a promoter that enables autonomous expression in a tetrad spore;
a sequence encoding an N-terminal or C-terminal fragment of a split marker protein;
a sequence encoding an interaction domain that permits association of the N-terminal and C-terminal fragments of the split marker protein to allow marker signal generation and detection; and optionally, a rare or unique restriction site.
   86. A chromosome of embodiment 85 wherein the promoter is pGPD1.
   87. A chromosome of embodiment 85 or 86 wherein the interaction domain is EF1 or EF2.
   88. A chromosome of any of embodiments 85-87 wherein the split marker is a drug resistance marker, a fluorescent protein or an auxotrophic marker.
   89. A chromosome of any of embodiments 85-88 wherein the restriction site is a homing endonuclease restriction site.
   90. A mating pair from a sporulating organism that utilizes meiosis in sexual reproduction, wherein each member of the mating pair includes a chromosome modified with a genetic construct encoding a marker.
   91. A mating pair of embodiment 90 wherein the marker is a drug resistance marker, a fluorescent protein, a cerulenin resistance marker or an auxotrophic marker.
   92. A mating pair of embodiment 90 or 91 wherein different chromosomes include different genetic constructs encoding different markers.
   93. A mating pair of embodiment 92 wherein the different markers are different drug resistance markers.
   94. A mating pair of embodiment 92 wherein the different markers are different fluorescent proteins.
   95. A mating pair of embodiment 92 wherein the different markers are different drug resistance markers and different fluorescent proteins.
   96. A mating pair of any of embodiments 90-95 wherein the genetic constructs include a promoter and/or a rare or unique restriction site.
   97. A mating pair of embodiment 96 wherein the promoter is pGPD1.
   98. A mating pair of embodiment 96 or 97 wherein the restriction site is a homing endonuclease restriction site.
   99. A mating pair from a sporulating organism that utilizes meiosis in sexual reproduction, wherein each chromosome is modified with a genetic construct encoding an aspect of a split marker.
   100. A mating pair of embodiment 99 wherein the aspect encoded by the genetic construct of one chromosome is an N-terminal fragment of a protein and the aspect encoded by the genetic construct of a second chromosome is a complementary C-terminal fragment of the protein.
   101. A mating pair of embodiment 99 or 100 wherein the genetic constructs include a promoter, a sequence encoding an interaction domain, and optionally a rare or unique restriction site.
   102. A mating pair of embodiment 101 wherein the promoter is pGPD1.
   103. A mating pair of embodiment 101 or 102 wherein the interaction domain is EF1 or EF2.
   104. A mating pair of any of embodiments 101-103 wherein the restriction site is a homing endonuclease restriction site.
   105. A mating pair from a sporulating organism that utilizes meiosis in sexual reproduction, wherein a chromosome of each member of the mating pair is modified with a genetic construct including:
a promoter that enables autonomous expression in a tetrad spore;
a sequence encoding an N-terminal or C-terminal fragment of a split marker protein;
a sequence encoding an interaction domain that permits association of the N-terminal and C-terminal fragments of the split marker protein to allow marker signal generation and detection; and
optionally, a rare or unique restriction site.
   106. A mating pair of embodiment 105 wherein the promoter is pGPD1.
   107. A mating pair of embodiments 105 or 106 wherein the interaction domain is EF1 or EF2.
   108. A mating pair of any of embodiments 105-107 wherein the split marker is a drug resistance marker, a fluorescent protein or an auxotrophic marker.
   109. A mating pair of any of embodiments 105-108 wherein the restriction site is a homing endonuclease restriction site.
   110. A mating pair wherein one member of the mating pair has a chromosome modified to controllably express an aspect of a split marker and the second member of the mating pair has a chromosome modified to controllably express a complementary aspect of the split marker.
   111. A kit for practicing a use or method of any of the preceding embodiments wherein the kit includes one or more of a fluorescent dye, a chromosome of any of embodiments 70-89, and/or a mating pair of any of embodiments 90-110.
   112. A kit for genetic mapping including a chromosome of any of embodiments 70-89, and/or a mating pair of any of embodiments 90-110.
   113. A chromosome of any of embodiments 70-89 derived from S. cerevisiae.
   114. A mating pair of any of embodiments 90-110 wherein the sporulating organism is S. cerevisiae.
   115. A method of capturing the tetrad relationship of recombinant progeny from a yeast cross using patterns of natural genetic sequences including:
obtaining genomic data from the recombinant progeny;
identifying a first set of tetrad relationships from centromere-flanking markers;
identifying a second set of tetrad relationships based on delta scores; and
outputting the first set of tetrad relationships and the second set of tetrad relationships.
   116. The method of embodiment 115, further including computing a significance cutoff for at least one of: pairs of recombinant progeny, triplets of recombinant progeny, or tetrads of recombinant progeny, the significance cutoff based on background noise.
   117. The method of embodiment 115 or 116, further including identifying a set of triplet relationships based on delta scores.
   118. The method of any of embodiments 115-117, further including identifying a set of pair relationships based on mutual information.
   119. The method of any of embodiments 115-118, wherein the tetrad relationship from the centromere-flanking markers includes a mirrored redundant-pattern in centromeric alleles.
   120. The method of any of embodiments 115-119, wherein the delta scores are calculated based on interaction information derived from analysis of tetrads of the recombinant progeny and of triplets of the recombinant progeny.
   121. A computer readable medium encoding computer-readable instructions that, when executed, cause one or more processors to perform the method of any of embodiments 115-120.
   122. A data-processing system including at least one processor and at least one data storage system, the at least one data storage system including computer-readable instructions that, when executed by the at least one processor, cause the data-processing system to perform the method of any of embodiments 115-120.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in the ability to—within the appropriate context—(1) sort tetrads from vegetative cells, dyads and dead cells; (2) identify recombinant progeny originating from spores of the same tetrad; or (3) identify a genetic recombination event in an offspring in a genomic region of interest.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

### ADDITIONAL REFERENCES

Koenraadt et al., Mol. Plant Pathol. 82:1348-1354, 1992
Winge & Laustsen, Physiol. 24: 263-315, 1937

1. A method of performing genetic analysis of unmodified cells, comprising
incubating a mixture of tetrads, vegetative cells, dyads, and dead cells in a fluorescent vital dye solution to produce a stained mixture of cells;
sorting the mixture of stained cells based on an optical characteristic attributable to the fluorescent vital dye to enrich for tetrads utilizing FACS-based sorting;
disrupting the original spore relationships of enriched tetrads;
identifying the original spore relationships of the enriched tetrads by
sequencing aspects of the natural genetic sequence of the spores, wherein such sequencing comprises sequencing less than 20% of the whole genome; and
grouping spores into tetrad relationships based on redundant and mirrored features in the natural genetic sequence of the spores.

2-3. (canceled)

4. The method of claim 1, wherein the vital dye is selected from Bis-(1,3-dibutylbarbituric acid) pentamethine oxonol (DiBac₄(5)); Anaspec AS-84701, calcein AM, carboxyfluorescein diacetate, copper phthalocyanine tetrasulfonate, DiOC (3,3′-dihexyloxacarbocyanine iodide), Evans blue, gadolinium texaphyrin, indocyanine green monosodium salt, isosulfan, methylene blue, Nile red, patent blue V, patent blue VF, propodium iodide, rhodamine 123, and sulfobromophthaleine.

5. The method of claim 1, wherein the vital dye is pentamethine oxonol or propodium iodide.

6. The method of claim 1 wherein the FACS-based sorting:
utilizes fluorescence intensity to sort tetrads, dyads, and dead cells away from live vegetative cells; and/or
utilizes 488 nm emission and a 595LP 610/20 filter; and/or
gates the tetrad, dyad, and dead cell population using forward scatter.

7-8. (canceled)

9. The method of claim 1 wherein the aspects of the natural genetic sequence comprise centromere-linked markers; allele presence; and/or location and/or number of recombination events.

10. The method of claim 1 wherein the sequencing is whole genome sequencing or restriction-associated DNA (RAD) sequencing.

11. The method of claim 1 wherein the sequencing comprises sequencing less than 10% of the whole genome; or less than 5% of the whole genome.

12. The method of claim 1 wherein the sequencing comprises sequencing 3% of the whole genome.

13. The method of claim 1 wherein grouping spores into tetrad relationships requires one or more of:
at least 50% shared valid markers flanking centromeres; and/or
at least 50% shared valid markers flanking centromeres and perfect consensus between these markers.

14. (canceled)

15. The method of claim 1 further comprising on or more of refining the grouping:
utilizing mutual information between two or more of the spores;
utilizing delta scores; and/or
calculating a pair-wise score.

16-17. (canceled)

18. The method of claim 1, further comprising computing a significance cutoff for at least one of: pairs of spores, triplets of spores, or tetrads of spores, the significance cutoff based on background noise.

19. The method of claim 15, further comprising identifying a set of triplet relationships based on delta scores.

20-27. (canceled)

28. A method of capturing the tetrad relationship of unmodified recombinant progeny from a yeast cross using patterns of natural genetic sequences comprising:
sequencing aspects of the natural genetic sequence of the recombinant progeny, wherein such sequencing comprises sequencing less than 20% of the whole genome; and
grouping recombinant progeny into tetrad relationships based on redundant and mirrored features in the natural genetic sequence of the grouped recombinant progeny.

29. The method of claim 28 wherein:
the aspects of the natural genetic sequence comprise one or more of:
centromere-linked markers; allele presence; and/or location and/or number of recombination events;
the sequencing is restriction-associated DNA (RAD) sequencing;
the sequencing comprises sequencing less than 10% of the whole genome;
the sequencing comprises sequencing less than 5% of the whole genome;
grouping recombinant progeny into tetrad relationships requires at least 50% shared valid markers flanking centromeres; and/or
grouping recombinant progeny into tetrad relationships requires at least 50% shared valid markers flanking centromeres and perfect consensus between these markers.

30-31. (canceled)

32. The method of claim 28 wherein the sequencing comprises sequencing 3% of the whole genome.

33-34. (canceled)

35. The method of claim 28 further comprising one or more of refining the grouping:
utilizing mutual information between two or more of the recombinant progeny;
utilizing delta scores;
by calculating a pair-wise score.

36-37. (canceled)

38. A method of capturing the tetrad relationship of unmodified recombinant progeny from a yeast cross using patterns of natural genetic sequences comprising:
obtaining genomic data from the recombinant progeny;
identifying a first set of tetrad relationships from centromere-flanking markers;
identifying a second set of tetrad relationships based on delta scores; and
outputting the first set of tetrad relationships and the second set of tetrad relationships.

39. The method of claim 38, further comprising one or more of:
computing a significance cutoff for at least one of:
pairs of recombinant progeny, triplets of recombinant progeny, or tetrads of recombinant progeny, the significance cutoff based on background noise;
identifying a set of triplet relationships based on delta scores;
identifying a set of pair relationships based on mutual information.

40-41. (canceled)

42. The method of claim 38, wherein:
the tetrad relationship from the centromere-flanking markers comprises a mirrored redundant-pattern in centromeric alleles; and/or
the delta scores are calculated based on interaction information derived from analysis of tetrads of the recombinant progeny and of triplets of the recombinant progeny.

43. (canceled)
